SEQUENCE LISTING (16 sequences)

SEQ ID NO: 1 - protein, 319 aa, artificial
  amino acids 25-335 of Homo sapiens G-CSFR (hG-CSFR) with a C-terminal polyhistidine tag
ECGHISVSAPIVHLGDPITASCIIKQNCSHLDPEPQILWRLGAELQPGGRQQRLSDGTQESIITLPHLNHTQAFLSCALNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTERAPTHHHHHHHH

SEQ ID NO: 2 - protein, 118 aa, artificial
  VH of C1.2
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAMLGELGWFDPWGQGTLVTVSS

SEQ ID NO: 3 - protein, 107 aa, artificial
  VL of C1.2
DIQMTQSPSALSASVGDRVTITCRASQGISSYLNWYQQKPGKAPKLLIYYASNLQNGIPSRFSGSGSGTDFTLTISSLQPEDFATYHCQQSYSTPLTFGGGTNVEIR

SEQ ID NO: 4 - protein, 118 aa, artificial
  VH of C1.2G
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSS

SEQ ID NO: 5 - protein, 107 aa, artificial
  VL  of C1.2G
DIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIK

SEQ ID NO: 6 - protein, 5 aa, artificial
  HCDR1 of C1.2
LYWMG

SEQ ID NO: 7 - protein, 17 aa, artificial
  C1.2 HCDR2
SISSSGGVTPYADSVKG

SEQ ID NO: 8 - protein, 9 aa, artificial
  C1.2 HCDR3
LGELGWFDP

SEQ ID NO: 9 - protein, 11 aa, artificial
  C1.2 LCDR1
RASQGISSYLN

SEQ ID NO: 10 - protein, 6 aa, artificial
  C1.2 LCDR2
ASNLQN

SEQ ID NO: 11 - protein, 9 aa, artificial
  C1.2 LCDR3
QQSYSTPLT

SEQ ID NO: 12 - protein, 8 aa, artificial
  consensus sequence of HCDR3 of C1.2
  VARIANT (5)..(5): X is an amino acid selected from the group consisting of tryptophan, glutamine, methionine, serine, phenylalanine, glutamic acid and histidine
  VARIANT (6)..(6): X iis an amino acid selected from the group consisting of phenylalanine, tyrosine, methionine, serine, glycine and isoleucine
  VARIANT (7)..(7): X is an amino acid selected from the group consisting of aspartic acid, methionine, glutamine, serine, leucine, valine, arginine and histidine
  VARIANT (8)..(8): X is an amino acid selected from the group consisting of proline gltuamic acid, alanine, leucine, phenylalanine, tyronis, threonine, asparagine, aspartic acid, serine, glycine, arginine, lysine
LGELXXXX

SEQ ID NO: 13 - protein, 9 aa, artificial
  consensus sequence of LCDR3 of C1.2
  VARIANT (1)..(1): X is an amino acid selected from the group consisting of glutamine, glutamic acid, histidine, alanine or serine
  VARIANT (2)..(2): X is an amino acid selected from the group consisting of glutamine, valine, phenylalanine, asparagine and glutamic acid
  VARIANT (3)..(3): X ian amino acid selected from the group consisting of serine or glycine
  VARIANT (4)..(4): X is an amino acid selected from the group consisting of tryptophan, methionine, phenylalanine, tyrosine, isoleucine and leucine
  VARIANT (5)..(5): X is an amino acid selected from the group consisting of glutamic acid, methionine, glutamine, tryptophan, serine, valine, asparagine, glycine, alanine, arganine, histidine, tyrosine, lysine or threonine
  VARIANT (6)..(6): X is an amino acid selected from the group consisting of tyrosine, methionine, isoleucine or threonine
  VARIANT (7)..(7): X is an amino acid selected from the group consisting of proline, alanine, histidine, glycine and lysine
  VARIANT (8)..(8): X is an amino acid selected from the group consisting of leucine, glutamine, methionine, alanine, phenylalanine, isoleucine, lysine, histidine and glycine
  VARIANT (9)..(9): X is an amino acid selected from the group consisting of threonine, phenylalanine, tyrosine, methionine, lysine, serine, histidine, proline, tryptophan, isoleucine, glutamine, glycine and valine
XXXXXXXXX

SEQ ID NO: 14 - protein, 445 aa, artificial
  C1.2G heavy chain IgG4 with S241P mutation
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 214 aa, artificial
  C1.2G with kappa light chain
DIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 444 aa, artificial
  C1.2G heavy chain IgG4 with S241P mutation and lacking C-terminal lysine residue
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2022/0220209 A1
(43) Pub. Date: Jul. 14, 2022

(54) METHOD OF TREATING NEUTROPHILIC CONDITIONS

(71) Applicant: CSL Innovation Pty Ltd, Parkville, Victoria (AU)

(72) Inventors: Karen Lisa INGUANTI, Parkville, Victoria (AU); Jolanta AIREY, Parkville, Victoria (AU); Jagdev SIDHU, Parkville, Victoria (AU); Michael TORTORICI, Parkville, Victoria (AU); Theresa YURASZECK, Parkville, Victoria (AU)

(73) Assignee: CSL Innovation Pty Ltd, Parkville, Victoria (AU)

(21) Appl. No.: 17/299,590

(22) PCT Filed: Dec. 4, 2019
(86) PCT No.: PCT/AU2019/051325

§ 371 Date: Jun. 3, 2021

Related U.S. Application Data
(60) Provisional application No. 62/774,974, filed on Dec. 4, 2018.
(60) Provisional application No. 62/885,373, filed on Aug. 12, 2019.
(60) Provisional application No. 62/897,487, filed on Sep. 9, 2019.

(51) Int. Cl. C07K 16/28; A61P 17/10; A61P 17/06

(52) U.S. Cl. CPC C07K 16/2866; A61K 2039/505; A61P 17/06; A61P 17/10

(57) ABSTRACT

The present disclosure relates to a method for reducing circulating neutrophils in a subject without causing sustained grade 3 or grade 4 neutropenia. The present disclosure also relates to methods for treating neutrophilic conditions with an antibody that inhibit G-CSF signalling. In particular, the present disclosure relates to methods of 5 treating neutrophilic dermatoses, such as hidradenitis suppurativa (HS) and palmoplantar pustulosis (PPP).

### RELATED APPLICATION DATA

This application claims priority from U.S. Patent Application No. 62/774,974 filed on 4 Dec. 2018 and entitled “Method of treating neutrophilic conditions”, U.S. Patent Application No. 62/885,373 filed on 12 Aug. 2019 and entitled “Method of treating neutrophilic conditions”, and U.S. Patent Application No. 62/897,487 filed on 9 Sep. 2019 and entitled “Method of treating neutrophilic conditions”. The entire contents of these applications are hereby incorporated by reference.

### FIELD

The present disclosure relates to a method for treating a neutrophilic condition with an antibody that binds to granulocyte-colony stimulating factor receptor (G-CSFR).

### BACKGROUND

Granulocyte colony-stimulating factor (G-CSF) is a major regulator of granulocyte production. G-CSF is produced by bone marrow stromal cells, endothelial cells, macrophages, and fibroblasts, and production is induced by inflammatory stimuli. G-CSF acts through the G-CSF receptor (G-CSFR), which is expressed on early myeloid progenitors, mature neutrophils, monocytes/macrophages, T and B lymphocytes and endothelial cells. Mice deficient in G-CSF or the G-CSFR exhibit marked neutropenia, demonstrating the importance of G-CSF in steady-state granulopoiesis. G-CSF increases the production and release of neutrophils, mobilizes hematopoietic stem and progenitor cell, and modulates the differentiation, lifespan, and effector functions of mature neutrophils. G-CSF may also exert effects on macrophages, including expansion of monocyte/macrophage numbers, enhancement of phagocytic function, and regulation of inflammatory cytokine and chemokine production. G-CSF has also been shown to mobilize endothelial progenitor cells and induce or promote angiogenesis.

While G-CSF is used therapeutically, e.g., to treat neutropenia and/or mobilize hematopoietic stem cells, it also has negative actions in some conditions, e.g., inflammatory conditions and/or cancer. For example, administration of G-CSF exacerbates rheumatoid arthritis (RA), murine collagen-induced arthritis (CIA) and a passive transfer model of CIA in rats. G-CSF has been found in the serum and synovial fluid of RA patients. Furthermore, interleukin (IL)-1 and tumor necrosis factor α (TNFα), which are found at increased levels in patients suffering from RA, induce the production of G-CSF by human synovial fibroblasts and chondrocytes. Mice deficient in G-CSF are resistant to the induction of acute and chronic inflammatory arthritis.

G-CSF has also been shown to play a role in multiple sclerosis (MS). For example, G-CSF enhances adhesion of an auto-reactive T cell line model of MS to extracellular matrix as effectively as interferon γ and TNFα, which are known to exacerbate MS symptoms. Moreover, G-CSF deficient mice are resistant to development of experimental autoimmune encephalomyelitis (EAE).

G-CSF and G-CSFR have also been tied to cancer, with studies showing that this signaling pathway contributes to chemotherapy resistance, growth, survival, invasiveness and metastasis of various cancers. Moreover, G-CSF has been shown to induce angiogenesis, a process important in the development of solid tumors.

Removal of myeloid cells, including neutrophils and monocytes/macrophages, using adsorptive granulocyte and monocyte apheresis (GMA) has also been shown to be useful for treating numerous conditions. GMA is an extracorporeal treatment in which a subject's blood is pumped through a column of cellulose acetate beads and myeloid cells are removed. Currently, GMA is approved in Japan for treatment of ulcerative colitis, Crohn's disease and pustular psoriasis. Clinical efficacy has also been reported for dermatological diseases (e.g., pyoderma gangrenosum, Behcet's disease, generalized pustular psoriasis, psoriasis, Still's disease, Sweet disease, cutaneous allergic vasculitis and systemic lupus erythematosus) and other conditions such as arthritis and psoriatic arthritis (see, e.g., Kanekura, J. Dermatol., 45: 943-950, 2018). A disadvantage of GMA is that a subject must attend a hospital setting in which they undergo a leukapheresis session once or twice per week five to ten times with each session lasting about one hour. Such treatment is time consuming, uncomfortable for the patient and requires specialized equipment and trained staff to administer.

It will be clear to the skilled person from the foregoing, that there is a need in the art for methods that reduce the signaling of G-CSF through the G-CSFR, without inducing neutropenia.

### SUMMARY

In work leading up to the present disclosure, the inventors sought to identify a dosage of an anti-G-CSFR antibody that was able to reduce the number of circulating neutrophils in a subject without inducing severe neutropenia or without inducing severe neutropenia for an extended period. By reducing the number of circulating neutrophils, the inventors are able to treat neutrophil-mediated conditions. However, the inventors also recognized the importance of not inducing severe neutropenia for an extended period to avoid placing a subject at risk of, e.g., an infection. The inventors have identified a dose of an antibody that reduces circulating neutrophil numbers but does not cause severe neutropenia in a subject for an extended period.

Based on the findings by the inventors, the disclosure provides a method for reducing circulating neutrophils in a subject without causing grade 3 neutropenia or grade 4 neutropenia (or severe neutropenia) for greater than two consecutive days, the method comprising administering to the subject a dose of between 0.1 mg/kg and 1.0 mg/kg of a compound that inhibits G-CSF signalling, e.g., a protein-based inhibitor, such as a protein comprising a Fc region of an antibody, for example, an antibody that binds G-CSFR and inhibits G-CSF signaling.

In one example, administration of the antibody does not cause grade 3 neutropenia or grade 4 neutropenia (or severe neutropenia) in the subject for greater than three consecutive days.

In one example, administration of the antibody does not cause grade 3 neutropenia or grade 4 neutropenia (or severe neutropenia) in the subject for greater than four or five or six consecutive days.

In one example, administration of the antibody does not cause grade 3 neutropenia or grade 4 neutropenia (or severe neutropenia) in the subject for greater than seven consecutive days.

In one example, the disclosure provides a method for reducing circulating neutrophils in a human subject without causing sustained grade 3 or grade 4 neutropenia for greater than seven consecutive days, the method comprising administering to the subject a dose of between 0.1 mg/kg and 1.0 mg/kg of an antibody that inhibits G-CSF signaling.

In one example, the compound is an antibody that binds to G-CSF and inhibits G-CSF signalling. In one example, the compound is an antibody that binds to G-CSFR and inhibits G-CSF signaling. For example, the antibody binds to or specifically binds to G-CSFR and competitively inhibits the binding of antibody C1.2G (also referred to as CSL324 herein) comprising a heavy chain variable region (V_{H}) comprising a sequence set forth in SEQ ID NO: 4 and a light chain variable region (V_{L}) comprising a sequence set forth in SEQ ID NO: 5 to G-CSFR.

In one example, the subject suffers from a neutrophil-mediated condition. Accordingly, the disclosure also provides a method for treating a neutrophil-mediated condition, the method comprising administering to a subject suffering from the neutrophil-mediated condition a dose of between 0.1 mg/kg and 1.0 mg/kg of a compound that inhibits G-CSF signalling (as discussed above and herein), e.g., an antibody that binds to G-CSF or G-CSFR and inhibits G-CSF signaling. In one example, the antibody binds to or specifically binds to G-CSFR and competitively inhibits the binding of antibody C1.2G comprising a V_{H} comprising a sequence set forth in SEQ ID NO: 4 and a light chain variable region V_{L} comprising a sequence set forth in SEQ ID NO: 5 to G-CSFR.

In one example, administration of the antibody does not cause grade 3 neutropenia or grade 4 neutropenia (or severe neutropenia) in the subject for greater than two consecutive days.

In one example, administration of the antibody does not cause grade 3 neutropenia or grade 4 neutropenia (or severe neutropenia) in the subject for greater than three consecutive days.

In one example, administration of the antibody does not cause grade 3 neutropenia or grade 4 neutropenia (or severe neutropenia) in the subject for greater than four or five or six consecutive days.

In one example, administration of the antibody does not cause grade 3 neutropenia or grade 4 neutropenia (or severe neutropenia) in the subject for greater than seven consecutive days.

In one example, administration of the antibody does not cause sustained grade 3 or grade 4 neutropenia in the subject for greater than seven consecutive days.

In one example, administration of the compound or antibody does not induce grade 4 neutropenia. In another example, administration of the compound or antibody induces grade 4 neutropenia for more than 3 consecutive days in less than 10% of a population of subjects to which it is administered.

In one example, administration of the compound or antibody is not associated with an infection, e.g., a serious infection, such as a tuberculosis infection.

In one example, administration of the compound or antibody does not induce neutropenia or induces grade 2 or grade 3 neutropenia for less than two consecutive days. For example, administration of the antibody induces grade 2 or grade 3 neutropenia for 36 hours or less, for example, for 24 hours or less.

In one example, administration of the compound or antibody does not induce neutropenia. Thus, in one example, the subject's absolute neutrophil count (ANC) remains above about 2×10⁹/L during treatment with the compound or antibody that inhibits G-CSF signalling.

In one example, the neutropenia is not associated with a fever.

In one example, the neutropenia is resolved without treatment.

In one example, the neutropenia is not associated with an infection, e.g., a serious infection, such as a tuberculosis infection.

In one example, administration of the compound or antibody does not induce grade 4 neutropenia following a single administration. In another example, administration of the compound antibody does not induce grade 4 neutropenia following multiple administrations, e.g., two administrations or three administrations or four administrations or five administrations or six administrations. In one example, administration of the compound or antibody does not induce grade 4 neutropenia following at least three administrations.

In one example, administration of the compound or antibody induces grade 2 or grade 3 neutropenia for less than two consecutive days following a single administration. In another example, administration of the compound or antibody induces grade 2 or grade 3 neutropenia for less than two consecutive days following multiple administrations, e.g., two or three administrations or four administrations or five administrations or six administrations. In one example, administration of the compound or antibody induces grade 2 or grade 3 neutropenia for less than two consecutive days following at least three administrations.

In one example, the compound or antibody is administered at a dose of between 0.1 mg/kg and 1 mg/kg. For example, the compound or antibody is administered at a dose of between 0.1 mg/kg and 0.9 mg/kg, for example, between 0.1 mg/kg and 0.8 mg/kg, for example between 0.1 mg/kg and 0.8 mg/kg. In one example, the compound or antibody is administered at a dose of between 0.1 mg/kg and 0.6 mg/kg. In one example, the compound or antibody is administered at a dose of between 0.3 mg/kg and 0.6 mg/kg.

In one example, the compound or antibody is administered at a dose of about 0.1 mg/kg.

In one example, the compound or antibody is administered at a dose of about 0.3 mg/kg.

In one example, the compound or antibody is administered at a dose of about 0.6 mg/kg.

In one example, the compound or antibody is administered multiple times. For example, the compound or antibody is administered once every 7 to 35 days. For example, the compound or antibody is administered every 14 to 28 days. For example, the compound or antibody is administered every 20 to 25 days. For example, the compound or antibody is administered multiple times, wherein the compound or antibody is administered once every 21 days. In this regard “every 21 days” (or any other number) will be understood by the skilled person to mean that the subsequent administration is performed on the 21^{st} day following the immediately prior administration.

The compound or antibody can be administered chronically, e.g., for months or years and the present disclosure is not limited to a specific time period unless stated otherwise.

In one example, the compound or antibody is administered until the condition or symptoms of the condition are resolved or managed.

In one example, the compound or antibody is administered to induce remission of a condition. In another example, the compound is administered to maintain remission of a condition.

In one example, one or more loading doses of the compound is administered followed by one or more maintenance doses. Generally, the loading doses will be higher or administered with a shorter time period between them than the maintenance doses.

In one example, the compound or antibody binds to an epitope comprising residues within one or two or three or four regions selected from 111-115, 170-176, 218-234 and/or 286-300 of SEQ ID NO: 1.

In one example, the antibody comprises:

(i) a V_{H} comprising an amino acid sequence set forth in SEQ ID NO: 4 and a V_{L} comprising an amino acid sequence set forth in SEQ ID NO: 5;
(ii) a V_{H} comprising an amino acid sequence set forth in SEQ ID NO: 2 and a V_{L} comprising an amino acid sequence set forth in SEQ ID NO: 3;
(iii) a V_{H} comprising three CDRs of a V_{H} comprising an amino acid sequence set forth in SEQ ID NO: 4 and a V_{L} comprising three CDRs of a V_{L} comprising an amino acid sequence set forth in SEQ ID NO: 5; or
(iv) a V_{H} comprising three CDRs of a V_{H} comprising an amino acid sequence set forth in SEQ ID NO: 2 and a V_{L} comprising three CDRs of a V_{L} comprising an amino acid sequence set forth in SEQ ID NO: 3.

In one example, the antibody comprises:

(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

In one example, the antibody comprises one heavy chain comprising a sequence set forth in SEQ ID NO: 14 and one heavy chain comprising a sequence set forth in SEQ ID NO: 16 and two light chains comprising a sequence set forth in SEQ ID NO: 15.

In one example, the antibody is administered in a composition comprising a mixture of the following antibodies:

(i) an antibody comprising a heavy chain comprising a sequence set forth in SEQ ID NO: 14 and a light chain comprising a sequence set forth in SEQ ID NO: 15;
(ii) an antibody comprising a heavy chain comprising a sequence set forth in SEQ ID NO: 16 and a light chain comprising a sequence set forth in SEQ ID NO: 15; and
(iii) an antibody comprises one heavy chain comprising a sequence set forth in SEQ ID NO: 14 and one heavy chain comprising a sequence set forth in SEQ ID NO: 16 and two light chains comprising a sequence set forth in SEQ ID NO: 15.

In some examples, the neutrophil-mediated condition is an autoimmune disease, an inflammatory disease, cancer or ischemia-reperfusion injury.

Exemplary autoimmune conditions include autoimmune intestinal disorders (such as Crohn's disease and ulcerative colitis), arthritis (such as rheumatoid arthritis, psoriatic arthritis and or idiopathic arthritis, e.g., juvenile idiopathic arthritis) or psoriasis.

Exemplary inflammatory conditions include inflammatory neurological conditions (e.g., Devic's disease, a viral infection in the brain, multiple sclerosis and neuromyelitis optica), an inflammatory lung disease (e.g., chronic obstructive pulmonary disease [COPD] or asthma) or an inflammatory eye condition (e.g., uveitis).

In one example, the neutrophil-mediated condition is ischemia-reperfusion injury. For example, the ischemia-reperfusion injury is due to or associated with tissue or organ transplantation (e.g., kidney transplantation). For example, the antibody is administered to a tissue or organ transplantation recipient, e.g., prior to organ collection and/or to a tissue or organ prior to transplantation or is administered to a harvested tissue or organ ex vivo.

In some examples, the neutrophil-mediated condition is psoriasis. In one example, the neutrophil-mediated condition is plaque psoriasis (also known in the art as “psoriasis vulgaris” or “common psoriasis”).

In one example, the neutrophil-mediated condition is a neutrophilic dermatosis or a neutrophilic skin lesion. For example, the neutrophilic dermatosis is a pustular psoriasis.

In one example, the neutrophilic dermatosis is selected from the group consisting of amicrobial pustulosis of the folds (APF); plaque psoriasis; CARD14-mediated pustular psoriasis (CAMPS); cryopyrin associated periodic syndromes (CAPS); deficiency of interleukin-1 receptor (DIRA); deficiency of interleukin-36 receptor antagonist(DIRTA); hidradenitis suppurativa (HS); palmoplantar pustulosis; pyogenic arthritis; pyoderma gangrenosum and acne (PAPA); pyoderma gangrenosum, acne, and hidradenitis suppurativa (PASH); pyoderma gangrenosum(PG); skin lesions of Behcet's disease; Still's disease; Sweet syndrome; subcorneal pustulosis (Sneddon-Wilkinson); pustular psoriasis; palmoplantar pustulosis; acute generalized exanthematic pustulosis; infantile acropustulosis; synovitis, acne, pustulosis; hyperostosis and osteitis (SAPHO) syndrome; bowel-associated dermatosis-arthritis syndrome (BADAS); neutrophilic dermatosis of the dorsal hands; neutrophilic eccrine hidradenitis; erythema elevatum diutinum; and Pyoderma gangrenosum. In one example, the neutrophilic dermatosis is hidradenitis suppurativa (HS) or palmoplantar pustulosis (PPP).

In one example, the neutrophilic dermatosis is hidradenitis suppurativa (HS). As shown in the Examples, it has been found that inhibition of G-CSF signalling significantly reduced neutrophil migration associated with CXCR1, which is a migratory chemokine receptor, the expression of which is correlated with HS disease severity.

In one example, the neutrophilic dermatosis is palmoplantar pustulosis (PPP). As shown in Example 5, it has been found that treatment of PPP with an antibody that inhibits G-CSF signalling is safe and efficacious.

Efficacy of treatment of PPP can be determined using any measure known in the art. For instance, in some examples, administration of an antibody as disclosed herein reduces a ppPASI score. The ppPASI is an assessment tool based on the Psoriasis Area and Severity Index that is widely used for assessing severity of chronic plaque psoriasis. Parameters including severity, erythema, total number of pustules and desquamation are scored on a scale of 1-4, then corrected for area and site involved (palm or sole). The sum of the four values produces the final ppPASI which ranges between 0 (no PPP) and 72 (the most severe PPP). ppPASI can be assessed at screening, prior to, during, and/or after administration an antibody disclosed herein to assess the efficacy of treatment. For example, a lower ppPASI score after administration of the antibody, relative to before administration, is evidence of effective treatment of PPP.

Another measure for assessing efficacy of treatment of PPP is Palm-Sole Physician Global Assessment (PGA). In one example, administration of an antibody as disclosed herein reduces the Palm-Sole Physician Global Assessment (PGA) score. The PGA is an average assessment of all psoriatic lesions based on erythema, scale, and induration. PGA can be assessed at screening, prior to, during, and/or after administration an antibody disclosed herein to assess the efficacy of treatment. For example, a lower PGA score after administration of the antibody, relative to before administration, is evidence of effective treatment of PPP. Other suitable measures for assessing efficacy of treating neutrophilic dermatoses such as PPP are described herein.

In some examples, the subject was diagnosed with PPP at least 1 year, or at least 2 years, or at least 3 years, or at least 4 years prior to treatment with the antibody that inhibits G-CSF signalling.

In some examples, the subject with PPP has been previously treated for PPP. In some examples, the subject has been previously treated with any one or more of the following therapies:
(i) methotrexate;
(ii) acitretin;
(iii)tacrolimus;
(iv) corticosteroids; and
(v) vitamin D and corticosteroids.

In some examples, the subject with PPP has a Palmoplantar Pustular Psoriasis Area Severity Index (ppPASI) of at least 11, or at least 16, or at least 21, or at least 26, or at least 31, prior to treatment with the antibody that inhibits G-CSF signalling. Thus, in some examples the PPP is moderate or severe PPP. In some examples, the PPP is severe PPP (i.e., a ppPASI of at least 16).

In some examples, the subject with PPP has a PPP-Physician's Global Assessment (PPP-PGA) score of 3 (i.e., “moderate”) or 4 (i.e., “severe”), prior to treatment with the antibody that inhibits G-CSF signalling.

In one example, the present disclosure provides a method for treating a neutrophilic dermatosis, the method comprising administering to a subject suffering from a neutrophilic dermatosis a dose of 0.1 to 1 mg/kg of an antibody that binds to or specifically binds to granulocyte-colony stimulating factor receptor (G-CSFR), wherein the antibody is administered multiple times once every 21 days and wherein the antibody comprises:

(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

In one example, the present disclosure provides a method for treating a neutrophilic dermatosis, the method comprising administering to a subject suffering from a neutrophilic dermatosis a dose of 0.1 to 1 mg/kg of an antibody that binds to or specifically binds to granulocyte-colony stimulating factor receptor (G-CSFR), wherein the antibody is administered multiple times once every 21 days and wherein the antibody comprises:

(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

In one example, the present disclosure provides a method for treating HS, the method comprising administering to a subject suffering from a neutrophilic dermatosis a dose of 0.1 to 1 mg/kg of an antibody that binds to or specifically binds to granulocyte-colony stimulating factor receptor (G-CSFR), wherein the antibody is administered multiple times once every 21 days and wherein the antibody comprises:

(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

In one example, the present disclosure provides a method for treating PPP, the method comprising administering to a subject suffering from a neutrophilic dermatosis a dose of 0.1 to 1 mg/kg of an antibody that binds to or specifically binds to granulocyte-colony stimulating factor receptor (G-CSFR), wherein the antibody is administered multiple times once every 21 days and wherein the antibody comprises:

(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

The present disclosure additionally provides a kit packed with an antibody as described herein packaged with instructions for use in a method described herein.

### BRIEF DESCRIPTION OF DRAWINGS

FIG. 1 is a graph which illustrates mean serum CSL324 concentration over time in healthy subjects administered a single dose of 0.1, 0.3, 0.6, 0.8, and 1.0 mg/kg C1.2G, as described in Example 1.

FIG. 2 is a graph which illustrates percent occupied target receptor (G-CSFR) over time in healthy subjects administered a single dose of 0.1, 0.3, 0.6, 0.8, and 1.0 mg/kg CSL324, as described in Example 1.

FIG. 3 is a schematic illustration detailing the screening, treatment and follow up periods for each cohort of subjects administered with CSL324 for the treatment of neutrophilic dermatosis, as described in Example 2.

FIG. 4 is a schematic detailing the lead in for Cohort #1 and the delayed start of Cohort #2 for subjects administered with CSL324 for the treatment of neutrophilic dermatosis, as described in Example 2.

FIG. 5 is a heatmap indicating absolute neutrophil count (ANC) according to neutropenia toxicity grade (i.e., Grades 1, 2, 3, and 4) in healthy subjects administered a single dose of 0.1, 0.3, 0.6, 0.8, and 1.0 mg/kg CSL324, as described in Example 1.

FIG. 6 is a heatmap indicating absolute neutrophil count (ANC) according to neutropenia toxicity grade (i.e., Grades 1, 2, 3, and 4) in healthy subjects administered three doses of 0.6 mg/kg CSL324, as described in Example 1.

FIG. 7 shows graphs illustrating the expression of CXCR1, a chemokine receptor associated with cell migration, on neutrophils from HS patients. FIG. 7A shows that CXCR1 expression was significantly higher in HS patient sample neutrophils compared to healthy controls. FIG. 7B shows the correlation between HS patient abscess and nodule count and CXCR1 expression on neutrophils in HS patients.

FIG. 8 shows graphs illustrating the effect of CSL324 on G-CSF-induced CXCR1 (FIG. 8A) and CXCR2 (FIG. 8B) expression on neutrophils. CSL324 (grey) did not alter the expression of either CXCR1 or CXCR2 compared to media alone, in the absence of G-CSF. Culture of neutrophils in the presence of G-CSF alone (black) increased the cell surface expression of CXCR1 and CXCR2 compared to media alone. Pre-incubation with CSL324 (grey) was able to reduce the G-CSF induced up-regulation of CXCR1 and CXCR2 expression.

FIG. 9 shows graphs illustrating the effect of CSL324 on G-CSF-induced neutrophil migration. Pre-incubation in the presence of G-CSF alone induced migration of neutrophils to MIP-2 (FIG. 9A; black bars), which was reduced to the same levels as the media alone control by CSL324 (FIG. 9A; grey bars). Pre-incubation with G-CSF resulted in up-regulation of CXCR1 and CXCR2 that correlated with increased migration of neutrophils to MIP-2 (FIGS. 9B and 9C).

FIG. 10 shows graphs illustrating the neutrophil count and expression of cell migration markers, CXCR1 and CXCR2, in psoriasis patients. Neutrophil counts (FIG. 10A) were significantly increased in the peripheral blood of people with psoriasis compared to unaffected controls. Stratification based on the severity of psoriasis as assessed by PASI score showed that neutrophil counts were significantly elevated in individuals with a PASI score of 10 or greater. The neutrophil:lymphocyte ratio (NLR) was significantly elevated in individuals with a PASI score of 10 or greater compared to individuals with a PASI score of less than 10 (FIG. 10B). Expression of CXCR2 was significantly elevated on the surface of neutrophils in both mild (PASI <10) and severe (PASI >10) psoriasis (FIG. 10 C). No statistically significant alteration in the levels of the chemokine receptor CXCR1 was detected (FIG. 10D).

FIG. 11 is a graph showing the Palmoplantar Pustular Psoriasis Area Severity Index (ppPASI) of a human subject with palmoplantar pustulosis (PPP) treated with five IV infusions of CSL324 every 21 days.

FIG. 12 is a graph showing the absolute neutrophil count (ANC) of a human subject with palmoplantar pustulosis (PPP) treated with five IV infusions of CSL324 every 21 days. Vertical dotted lines indicate the CSL324 dosages. The lower limit and upper limit of the normal ANC range are indicated by “LLN” and “ULN” respectively.

### KEY TO SEQUENCE LISTING

SEQ ID NO: 1—amino acids 25-335 of Homo sapiens G-CSFR (hG-CSFR) with a C-terminal polyhistidine tag

### SEQ ID NO: 2—VH of C1.2

### SEQ ID NO: 3—VL of C1.2

### SEQ ID NO: 4—VH of C1.2G

### SEQ ID NO: 5—VL of C1.2G

### SEQ ID NO: 6—HCDR1 of C1.2

### SEQ ID NO: 7—HCDR2 of C1.2

### SEQ ID NO: 8—HCDR3 of C1.2

### SEQ ID NO: 9—LCDR1 of C1.2

### SEQ ID NO: 10—LCDR2 of C1.2

### SEQ ID NO: 11—LCDR3 of C1.2

SEQ ID NO: 12—consensus sequence of HCDR3 of C1.2
SEQ ID NO: 13—consensus sequence of LCDR3 of C1.2
SEQ ID NO: 14—Heavy chain of C1.2G with stabilized IgG4 constant region
SEQ ID NO: 15—Light chain of C1.2G with kappa constant region
SEQ ID NO: 16—Heavy chain of C1.2G with stabilized IgG4 constant region and lacking C-terminal lysine.

### DESCRIPTION OF EMBODIMENTS

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

Reference herein to “granulocyte colony-stimulating factor” (G-CSF) includes native forms of G-CSF, mutant forms thereof, e.g., filgrastim and pegylated forms of G-CSF or filgrastim. This term also encompasses mutant forms of G-CSF retaining activity to bind to G-CSFR (e.g., human G-CSFR) and induce signaling.

G-CSF is a major regulator of granulocyte production. G-CSF is produced by bone marrow stromal cells, endothelial cells, macrophages, and fibroblasts, and production is induced by inflammatory stimuli. G-CSF acts through the G-CSF receptor (G-CSFR), which is expressed on early myeloid progenitors, mature neutrophils, monocytes/macrophages, T and B lymphocytes and endothelial cells.

For the purposes of nomenclature only and not limitation, an exemplary sequence of a human G-CSFR is set out in NCBI Reference Sequence: NP_000751.1 (and set out in SEQ ID NO: 16). The sequence of G-CSFR from other species can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)) Reference to human G-CSFR may be abbreviated to hG-CSFR and reference to cynomolgus monkey G-CSFR may be abbreviated to cynoG-CSFR. Reference to soluble G-CSFR refers to polypeptides comprising the ligand binding region of G-CSFR. The Ig and CRH domains of the G-CSFR are involved in ligand binding and receptor dimerization (Layton et al., J. Biol Chem., 272: 29735-29741, 1997 and Fukunaga et al, EMBO J. 10: 2855-2865, 1991). Soluble forms of G-CSFR comprising these portions of the receptor have been used in various studies of the receptor and mutation of the free cysteines at positions 78, 163, and 228 of the receptor assists in expression and isolation of the soluble receptor polypeptide (Mine et al., Biochem., 43: 2458-2464 2004) without affecting ligand binding.

As used herein, the term “neutrophil-mediated condition” will be understood to encompass any adverse condition or disease that is caused by the activity of neutrophils or for which therapeutic benefit is achieved by removal of or reduction in the number of circulating neutrophils.

As used herein, the term “neutropenia” is used to refer to an absolute neutrophil count (ANC) below the lower limit of normal range, for example an ANC of less than 2000 cells/μL blood, or less than 1500 cells/μL blood, or less than 1000 cells/μL blood, for example less than 500 cells/μL blood (see Sibille et al. 2010 Br J Clin Pharmacol 70(5): 736-748). In some examples, the antibody that inhibits G-CSF signaling is administered in an amount that does not cause severe neutropenia. As used herein, the term “severe neutropenia” is used to refer to an absolute neutrophil count (ANC) of less than 1000 cells/μL blood. For the purposes of the present disclosure, the following will be used to define the grades of neutropenia
Grade 1: <2.0×10⁹/L (<2000/mm³) and >1.1×10⁹/L (>1500/mm³)
Grade 2: <1.5×10⁹/L (<1500/mm³) and >1.0×10⁹/L (>1000/mm³)
Grade 3: <1.0×10⁹/L (<1000/mm³) and >0.5×10⁹/L (>500/mm³)
Grade 4: <0.5×10⁹/L (<500/mm³).

As used herein, the terms “preventing”, “prevent” or “prevention” include administering an antibody of the disclosure to thereby stop or hinder the development of at least one symptom of a condition. This term also encompasses treatment of a subject in remission to prevent or hinder relapse. For example, a subject suffering from relapsing-remitting multiple sclerosis is treated during remission to thereby prevent a relapse.

As used herein, the terms “treating”, “treat” or “treatment” include administering an antibody described herein to thereby reduce or eliminate at least one symptom of a specified disease or condition.

As used herein, the term “subject” shall be taken to mean any animal including humans, for example a mammal Exemplary subjects include but are not limited to humans and non-human primates. For example, the subject is a human.

The skilled artisan will be aware that an “antibody” is generally considered to be an antibody that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., PR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

As used herein, the term “binds” in reference to the interaction of an antibody or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that an antibody of the disclosure reacts or associates more frequently, more rapidly, with greater duration and/or with greater affinity with a particular antigen or cell expressing same than it does with alternative antigens or cells. For example, an antibody binds to G-CSFR (e.g., hG-CSFR) with materially greater affinity (e.g., 20 fold or 40 fold or 60 fold or 80 fold to 100 fold or 150 fold or 200 fold) than it does to other cytokine receptor or to antigens commonly recognized by polyreactive natural antibodies (i.e., by naturally occurring antibodies known to bind a variety of antigens naturally found in humans) Generally, but not necessarily, reference to binding means specific binding, and each term shall be understood to provide explicit support for the other term.

As used herein, the term “epitope” (syn. “antigenic determinant”) shall be understood to mean a region of hG-CSFR to which an antibody binds. This term is not necessarily limited to the specific residues or structure to which the antibody makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when hG-CSFR is folded, i.e., a “conformational epitope”. For example, a conformational epitope comprises amino acids in one or more or two or more or all of the regions corresponding to 111-115, 170-176, 218-234 and/or 286-300 of SEQ ID NO: 1. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that an antibody of the disclosure (or an antigen binding site thereof) reduces or prevents binding of another antibody to G-CSFR, e.g., to hG-CSFR. This may be due to the antibody (or antigen binding site) and the other antibody binding to the same or an overlapping epitope. It will be apparent from the foregoing that the antibody need not completely inhibit binding of the other antibody, rather it need only reduce binding by a statistically significant amount, for example, by at least about 10% or 20% or 30% or 40% or 50% or 60% or 70% or 80% or 90% or 95%. Preferably, the antibody reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to G-CSFR either in the presence or absence of the antibody. If less antibody binds in the presence of the antibody than in the absence of the antibody, the antibody is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit an antibody (or antigen binding site thereof) that binds to one epitope to competitively inhibit the binding of an antibody (or antigen binding site) that binds to the other epitope. For example, the “overlapping” epitopes share at least 1 or 2 or 3 or 4 or 5 or 6 or 7 or 8 or 9 or 20 amino acids.

As used herein, the term “neutralize” shall be taken to mean that an antibody is capable of blocking, reducing or preventing G-CSF-mediated signaling in a cell through the G-CSFR. Methods for determining neutralization are known in the art and/or described herein.

### Treating Neutrophil-Mediated Conditions

In some examples, the neutrophil-mediated condition is an autoimmune disease, an inflammatory disease, cancer or ischemia-reperfusion injury.

Exemplary autoimmune conditions include autoimmune intestinal disorders (such as Crohn's disease and ulcerative colitis), arthritis (such as rheumatoid arthritis, psoriatic arthritis and or idiopathic arthritis, e.g., juvenile idiopathic arthritis) or psoriasis.

Exemplary inflammatory conditions include inflammatory neurological conditions (e.g., Devic's disease, a viral infection in the brain, multiple sclerosis and neuromyelitis optica), an inflammatory lung disease (e.g., chronic obstructive pulmonary disease [COPD] or asthma) or an inflammatory eye condition (e.g., uveitis).

In one example, the neuthrophil mediated condition is ischemia-reperfusion injury. For example, the ischemia-reperfusion injury is due to or associated with tissue or organ transplantation (e.g., kidney transplantation). For example, the antibody is administered to a tissue or organ transplantation recipient, e.g., prior to organ collection and/or to a tissue or organ prior to transplantation or is administered to a harvested tissue or organ ex vivo.

In one example, the neutrophil-mediated condition is a neutrophilic dermatosis or a neutrophilic skin lesion.

### Exemplary Autoimmune Conditions

In one example, the neutrophil-mediated condition is rheumatoid arthritis (RA). Certain subtypes of RA may be treated in accordance with the present disclosure. In one instance, moderate to severe RA, is treated by administering an antibody disclosed herein. In one example, the subject has confirmed moderate to severe RA, e.g., polyarticular RA.

The present disclosure also provides a method for treating certain subpopulations of RA patients who may be especially difficult to treat. For example, in one instance, the present disclosure provides a method for treating patients who have a subtherapeutic response to a therapy, such as those who have been unresponsive or intolerant to methotrexate or an inhibitor of tumor necrosis factor for treatment for their RA.

The present disclosure also provides methods for improving RA symptoms in a subject based on indices used to measure the disease state. Treatment of RA using an antibody disclosed herein may also be determined using measures known in the art.

Methods for measuring the severity of RA will be apparent to the skilled artisan. For example, comparing the number of tender and swollen joints between baseline and various time points during treatment is a typical way to assess joint status and response to treatment. In the American College of Rheumatology (ACR) joint count for RA (Felson et al. Arthritis & Rheumatology 38: 727-735, 1995), 68 joints are assessed for tenderness and 66 for swelling (the hip is not assessed for swelling). In the Disease Activity Score (DAS) employed primarily in Europe, either a 44- or 28-joint count is used in RA. In addition to the joint count, the ACR evaluation criteria include the following elements to comprise a composite score: patient global (on a visual analog scale [VAS]), patient pain, physician global, Health Assessment Questionnaire (HAQ; a measure of function), and an acute-phase reactant (either C-reactive protein or sedimentation rate). An ACR 20 response would constitute a 20% improvement in tender and swollen joint count and a 20% improvement of at least 3 of the other 5 elements in the composite criteria. ACR 50 and 70 responses represent at least a 50% and 70% improvement of these elements. The ACR system only represents change, whereas the DAS system represents both current state of disease activity and change. The DAS scoring system uses a weighted mathematical formula, derived from clinical trials in RA. For example, the DAS 28 is 0.56(T28)+0.28(SW28)+0.70(Ln ESR)+0.014 GH wherein T represents tender joint number, SW is swollen joint number, ESR is erythrocyte sedimentation rate, and GH is global health. Various values of the DAS represent high or low disease activity as well as remission, and the change and endpoint score result in a categorization of the patient by degree of response (none, moderate, good).

In one example, the neutrophil-mediated condition is psoriasis. As used herein, the term “psoriasis” encompasses all subtypes of psoriasis, including plaque, guttate, inverse, pustular, and erythrodermic. In one example, the neutrophil-mediated condition is plaque psoriasis (also known in the art as “psoriasis vulgaris” or “common psoriasis”). Certain subtypes of psoriasis may be treated in accordance with the present disclosure. In one instance, moderate to severe psoriasis, is treated by administering an antibody disclosed herein. In one example, the subject has confirmed moderate to severe psoriasis, e.g., chronic moderate to severe psoriasis.

The present disclosure also provides a method for treating certain subpopulations of psoriasis patients who may be especially difficult to treat. For example, in one instance, the present disclosure provides a method for treating patients who have a subtherapeutic response to a therapy, such as those who have been unresponsive or intolerant to topical corticosteroids or an inhibitor of tumor necrosis factor for treatment for their psoriasis.

The present disclosure also provides methods for improving psoriasis symptoms in a subject based on indices used to measure the disease state. Treatment of psoriasis using an antibody disclosed herein may also be determined using measures known in the art.

Methods for measuring the severity of psoriasis will be apparent to the skilled artisan. For example, the Psoriasis Area and Severity Index (PASI) is used by dermatologists to assess psoriasis disease intensity. This index is based on the quantitative assessment of three typical signs of psoriatic lesions: erythema, infiltration, and desquamation, combined with the skin surface area involvement. Since its development in 1978, this instrument has been used throughout the world by clinical investigators (Fredriksson T, Petersson U: Dermatologica 1978; 157: 238-41). PASI is indicated as PASI 50 (a 50 percent improvement in PASI from baseline), PASI 75 (a 75 percent improvement in PASI from baseline), PASI 90 (a 90 percent improvement in PASI from baseline), and PASI 100 (a 100 percent improvement in PASI from baseline).

The Physicians Global Assessment (PGA) is used to assess psoriasis activity and follow clinical response to treatment. It is a six-point score that summarizes the overall quality (erythema, scaling and thickness) and extent of plaques relative to the baseline assessment. A patient's response is rated as worse, poor (0-24%), fair (25-49%), good (50-74%), excellent (75-99%), or cleared (100%) (van der Kerkhof P. Br J Dermatol 137:661-662, 1997). Other measures of improvements in the disease state of a subject having psoriasis include clinical responses, such as the Dermatology Life Quality Index (DLQI) and the Minimum Clinically Important Difference (MCID), described in more detail below.

### Asthma

In one example, the neutrophil-mediated condition is asthma, e.g., severe asthma. In the context of asthma, the term “treating” or “treat” refers to administering an antibody described herein to reduce, eliminate, or prevent an occurrence or exacerbation of at least one symptom. For example, an antibody described herein can be administered in order to prevent an asthmatic attack. Alternatively, or additionally, the antibody can be administered to alleviate asthmatic symptoms such as wheezing, shortness of breath, chest tightness, and/or coughing.

In one example, the asthma is allergic asthma. As used herein, the term “allergic asthma” (also referred to as “acute asthma”) refers to asthma triggered by allergens (e.g., dust mite or pollen) activating mast cells located beneath the mucosa of the lower airways of respiratory tract. Activation of mast cells triggers release of granules that stimulate the nasal epithelium to produce mucus and subsequent contraction of smooth muscle within the airway. This contraction of smooth muscle constricts the airway, causing the asthmatic symptoms.

In one example, the asthma is neutrophilic asthma. As used herein, the term “neutrophilic asthma” refers to a subset of asthma that is characterized by an increase in the amount of neutrophils in the airways of a subject. Neutrophilic asthma can be categorized by high neutrophil counts in sputum, for example greater than 40% or greater than 60% of sputum cells. The response to treatment of neutrophilic asthma with corticosteroids is often found to be ineffective, compared to patients with eosinophilic asthma. Neutrophilic asthma is also associated with upregulated expression of IL-8, IL-17, and IFN-γ in the airways. In contrast, “eosinophilic asthma”, which is characterised by an increase in the levels of eosinophils in the airways, is associated with an increase in IL-5 expression and a Th2-dominant inflammatory response.

In one example, the asthma is mixed granulocytic asthma. As used herein, the term “mixed granulocytic asthma” refers to asthma which is characterized by an increase in the amount of both neutrophils and eosinophils in the airways of a subject.

In one example, the asthma is severe asthma. As used herein, the term “severe asthma”, refers to asthma for which symptoms are only partially controlled or even uncontrolled, despite intensive treatment with standard therapies. Severe asthma can be defined according the International ERS/ATS guidelines on definition, evaluation and treatment of severe asthma (Chung et al., Eur Respir J. 2014; 43(2):343-73). According to the ERS/ATS guidelines, severe asthma is defined as asthma which would require treatment with high dose inhaled corticosteroids (ICS) plus a second controller (eg a Long-acting 132 agonist, montelukast, or theophylline) and/or treatment with systemic corticosteroids to prevent it from becoming uncontrolled or which remains uncontrolled despite the treatment.

In one example, the asthma is moderate asthma. In one example, the asthma is moderate or severe asthma. Asthma is classified as “moderate” if symptoms occur daily, symptoms exacerbate frequently and usually last several days. Coughing and wheezing may disrupt normal daily activities and make it difficult to sleep. Nighttime symptom exacerbations occur more than once a week. In moderate asthma, lung function is roughly between 60% and 80% of normal, without treatment. The Global Initiative for Asthma (GINA) guidelines can be used to classify asthma severity, including moderate asthma.

In one example, the asthma is poorly controlled or uncontrolled asthma. The level of asthma control, as opposed to severity, can be determined using, for example, an Asthma Control Questionanaire (ACQ) as described in Juniper et al., (1999) Eur Respir J 14:902-907, Juniper et al., Respiratory Medicine (2006) 100:616-621, and Juniper et al., Respiratory Medicine (2005) 99:553-558.

In one example, the asthma is refractory asthma. As used herein, the term “refractory asthma” includes patients with “fatal” or “near fatal” asthma as well as the asthma subgroups such as “severe asthma” and “steroid-dependent and/or resistant asthma,” “difficult to control asthma,” “poorly controlled asthma,” “brittle asthma,” or “irreversible asthma.” Refractory asthma can be defined as per the American Thoracic Society guidelines when one or both major criteria and two minor criteria, described as follows, are fulfilled. The major criteria are: In order to achieve control to a level of mild-moderate persistent asthma: (1) Treatment with continuous or near continuous (>50% of year) oral corticosteroids 2) Requirement for treatment with high-dose inhaled corticosteroids. The minor criteria are: (1) Requirement for daily treatment with a controller medication in addition to inhaled corticosteroids e.g., LABA, theophylline or leukotriene antagonist (2) Asthma symptoms requiring short-acting β-agonist use on a daily or near daily basis (3) Persistent airway obstruction (FEV₁<80% predicted; diurnal peak expiratory flow (PEF) variability >20%) (4) One or more urgent care visits for asthma per year (5) Three or more oral steroid “bursts” per year (6) Prompt deterioration with ≤25% reduction in oral or inhaled corticosteroid dose (7) Near fatal asthma event in the past. For the purposes of definition of refractory asthma, the drug (μg/d) and the dose (puffs/d) are as follows: (a) Beclomethasone dipropionate >1,260>40 puffs (42 μg/inhalation) >20 puffs (84 μg/inhalation); (b) Budesonide >1,200 >6 puffs; (c) Flunisolide >2,000 >8 puffs; (d) Fluticasone propionate >880 >8 puffs (110 μg), >4 puffs (220 μg); (e) Triamcinolone acetonide >2,000 >20 puffs.

“Chronic asthma” is not caused by allergens, but rather a result of the inflammation obtained from acute asthma. Acute asthma causes chronic inflammation, which causes the mucosal epithelium to become hypersensitive to environmental responses. So simple environmental agents, such as smoke, can stimulate the hypersensitive epithelium to produce large amounts of mucous and constrict.

In one example, the antibody is administered in an amount sufficient to enhance lung function. Lung function can be assessed by, for example, spirometry. In one example, the antibody is administered in an amount sufficient to increase FEV₁ (forced expiratory volume in one second). In one example, the antibody is administered in an amount sufficient to increase FVC (forced vital capacity). The FEV₁ is the volume expired in the first second of maximal expiration initiated at full inspiration, and is one measure of lung function. FVC is the maximum volume of air that can be expired during the test.

In one example, the antibody is administered in an amount sufficient to reduce or prevent airway hyper-responsiveness (AHR). AHR is an increased sensitivity of the airways to an inhaled constrictor agonist, a steeper slope of the dose-response curve, and a greater maximal response to the agonist. AHR is generally associated with lower lung function and asthmatic symptoms. AHR can be assessed, for example, with a bronchial challenge test. This most often uses constrictor agonists like methacholine or histamine. These chemicals trigger bronchospasm in non-asthmatic subjects as well, but subjects with AHR have a lower response threshold to the constrictor agonists. Suitable methods are described in (FitzPatrick et al., Sci Rep, 2016 6:22751).

### Exemplary Neutrophilic Dermatoses

In one example, the neutrophil-mediated condition is HS. HS is a skin disorder of the apocrine glands (sweat glands found on certain parts of the body) and hair follicles in which swollen, painful, inflamed lesions or lumps develop in the groin and sometimes under the arms and under the breasts. HS occurs when apocrine gland outlets become blocked by perspiration or are unable to drain normally because of incomplete gland development. Secretions trapped in the glands force perspiration and bacteria into surrounding tissue, causing subcutaneous induration, inflammation, and infection. HS is confined to areas of the body that contain apocrine glands. These areas are the axillae, areola of the nipple, groin, perineum, circumanal, and periumbilical regions.

Certain subtypes of HS may be treated in accordance with the present disclosure. In one instance, moderate to severe HS, is treated by administering an antibody disclosed herein. In one example, chronic HS, e.g., moderate to severe chronic HS, is treated by administering an antibody disclosed herein. In one example, the subject has confirmed moderate to severe HS, e.g., confirmed chronic moderate to severe HS.

The present disclosure also provides a method for treating certain subpopulations of HS patients who may be especially difficult to treat. For example, in one instance, the present disclosure provides a method for treating patients who have a subtherapeutic response to a therapy, such as those who have been unresponsive or intolerant to oral antibiotics for treatment for their HS.

The present disclosure also provides methods for improving HS symptoms in a subject based on indices used to measure the disease state.

Treatment of HS using an antibody disclosed herein may also be determined using measures known in the art. Treatment of HS may be determined using any of the measures known in the art, e.g., improvement in Hurley Staging or the Sartorius scale, or any measure known to those in the art.

For example, in one instance, an improvement in the Hurley stage of the subject having HS, or any of the measures described herein, is evidence of effective HS treatment. In one instance, the severity of HS is determined according to the Hurley staging system. Hurley staging is based on assigning the subject having HS one of three different “Stages” depending on the disease level. More specifically, Stage I refers to abscess formation, single or multiple, without sinus tracts and cicatrisation; Stage II refers to recurrent abscesses with tract formation and cicatrisation, as well as single or multiple, widely separated lesions; and Stage III, which refers to diffuse or near-diffuse involvement, or multiple interconnected tracts and abscesses across the entire area. Hurley Stage III is the most severe form. In one instance, the subject having HS has HS lesions that are present in at least two distinct anatomic areas (e.g. left and right axilla; or left axilla and left inguinal-crural fold), one of which is at least Hurley Stage II. In another instance, the subject being treated has at least one lesion that is at least a Hurley Stage II.

In one instance, treatment of HS with an antibody disclosed herein is determined by an improved Hurley score relative to a given baseline, e.g., the Hurley stage of the subject prior to treatment with the TNFα inhibitor. In one instance, improvement in a Hurley score indicates that the Hurley score of the subject has either improved or been maintained following treatment with an antibody.

Severity of HS may be determined according to standard clinical definitions. See, for example, Hurley staging {III vs. (I or II)} for HS (Poli F, Jemec GBE, Revuz J., Clinical Presentation. In: Jemec GBE, Revuz J, Leyden J J, editors. Hidradenitis Suppurativa. Springer, New York, 2006, pp 11-24). Hurley stage III disease is the most severe stage of hidradenitis suppurativa, reflecting diffuse or near-diffuse involvement of affected areas.

In one example, the Sartorius scale may be used as an index for measuring efficacy of an antibody. The Sartorius scale is described by Sartorius et al. in British Journal of Dermatology, 149: 211-213. Briefly, the following outcome variables are explicitly mentioned in reports based on the Sartorius scale: (1) anatomical region involved (axilla, groin, gluteal or other region or inframammary region left and/or right: 3 points per region involved); (2) number and scores of lesions (abscesses, nodules, fistulas, scars: points per lesion of all regions involved: nodules 2; fistulas 4; scars 1; others 1); (3) the longest distance between two relevant lesions, i.e., nodules and fistulas, in each region, or size if only one lesion (<5 cm, 2; <10 cm, 4; >10 cm, 8); and (4) are all lesions clearly separated by normal skin? In each region (yes 0/no 6). By assigning numerical scores to these variables, disease intensity can be quantified in a more clinically meaningful way on an open-ended scale. A total score as well as scores of selected regions chosen for surgical or other intervention can be calculated and followed over time.

In one example, treatment of HS with an antibody disclosed herein is determined according to an achieving an HiSCR (Hidradenitis Suppurativa Clinical Response) of the subject being treated. The HisSCR is defined as at least a 50% reduction in the total inflammatory lesion (abscess and inflammatory nodule) count (AN count) in a subject relative to baseline, with no increase in abscess count and no increase in draining fistula count. In one instance, treatment of HS in a subject is defined as an at least 50% reduction in the inflammatory lesion (abscess and nodule) count. The HiSCR scoring system was designed to assess hidradenitis suppurativa activity in an affected subject before and after a treatment.

In another example, treatment of HS with an antibody disclosed herein is defined as achieving an Physician's Global Assessment (PGA) score as defined below, of clear (0), minimal (1), or mild (2), with an improvement (i.e., reduction) from baseline PGA score of at least 1 grade or 2 grades, optionally, at the end of a treatment period (such as week 16). The baseline PGA score is the PGA score measured just prior to the commencement of treatment, to which the PGA score obtained after a period of treatment is compared.

TABLE 1
PGA Scoring

| Score | Rating | Description |
|---|---|---|
| 0 | Clear | No abscesses, no draining |
| | | fistulas, no nodules |
| 1 | Minimal | No abscesses, no draining |
| | | fistulas, no inflammatory |
| | | nodules, presence of non- |
| | | inflammatory nodules |
| 2 | Mild | No abscesses or draining |
| | | fistulas, and less than 5 |
| | | inflammatory nodules, or |
| | | single abscess or draining |
| | | fistula, and no inflammatory |
| | | nodules |
| 3 | Moderate | No abscesses or draining |
| | | fistulas, and at least 5 |
| | | inflammatory nodules, or |
| | | single abscess or draining |
| | | fistula in the presence of |
| | | inflammatory nodules, or |
| | | between 2 and 5 abscesses or |
| | | draining fistulas with or |
| | | without inflammatory nodules, |
| | | up to 10 |
| 4 | Severe | Between 2 and 5 abscesses and |
| | | draining fistulas with or |
| | | without inflammatory nodules |
| | | that are greater than 10 |
| 5 | Very Severe | More than 5 abscesses or |
| | | draining fistulas |

In one instance, the present disclosure provides a method for improving the DLQI score of a subject suffering from HS. In one instance, the improvement in the DLQI score is determined by achieving a score, e.g., a statistically significant score, correlating with a “no” or “small impact” of the disease state on the subject.

In another example, treatment of HS with an antibody disclosed herein is defined as achieving International Hidradenitis Suppurativa Severity Score System (IHS4). The IHS4 is a validated tool for the dynamic severity assessment of HS (Zouboulis, et al., Br J Dermatol, 177: 1401-09, 2017) and improves upon the HiSCR assessment as it is designed to assess treatment response rather than disease severity cross-sectionality (Kimball et al., Br J Dermatol, 171: 1434-42, 2014). The IHS4 score (points)=(number of nodules multiplied by 1)+(number of abscesses multiplied by 2)+[number of draining tunnels (fistulae/sinuses) multiplied by 4]. A score of 3 or less signifies mild HS, a score of 4-10 signifies moderate HS and a score of 11 or higher signifies severe HS (Zouboulis, et al., Br J Dermatol, 177: 1401-09, 2017). In one example, the subject has an IHS4 score of ≥4 prior to treatment.

In one example, the present disclosure provides a method for decreasing the number of inflammatory lesions (AN count) in a subject having HS, said method comprising systemically administering an antibody disclosed herein to the subject, such that the AN count is decreased. The decrease in AN count may be anything greater than 10%, e.g., the AN count may be reduced by at least a 50% reduction in the subject relative to baseline AN count. The subject may also exhibit other improvements in HS following treatment with an antibody disclosed herein, for example the subject may have no increase in an abscess count and/or no increase in a draining fistula count following administration with the antibody.

In one example, In one example, the neutrophil-mediated condition is PPP. PPP is a chronic pustular condition affecting the hands and/or soles of the feet. PPP can occur with psoriasis or without any skin disease. PPP affects the eccrine sweat glands which are most common on the palms and soles. PPP presents as crops of itchy or sore pustules on the palms and/or soles. PPP can occur on one or both hands and/or feet. Scaly red patches may also be seen in association with the pustules. In the more chronic stages of the disease, the skin can be dry and thickened with deep fissures (cracks in the skin). There is usually a sharp demarcation between the normal and affected skin areas. PPP varies in severity and may persist for many years. The discomfort can be considerable, interfering with work and affecting quality of life. A form of PPP which affects the tips of the fingers is called acrodermatitis continua of Hallopeau or acropustulosis. It can lead to destruction of the fingernail on affected digits.

Certain subtypes of PPP may be treated in accordance with the present disclosure. In one instance, moderate to severe PPP, is treated by administering an antibody disclosed herein. In one example, chronic PPP, e.g., moderate to severe chronic PPP, is treated by administering an antibody disclosed herein. In one example, the subject has confirmed moderate to severe PPP, e.g., confirmed chronic moderate to severe PPP.

The present disclosure also provides a method for treating certain subpopulations of PPP patients who may be especially difficult to treat. For example, in one instance, the present disclosure provides a method for treating patients who have a subtherapeutic response to a therapy, such as those who have been unresponsive or topical corticosteroids, vitamin D3 analogues, etretinate, and phototherapy for treatment for their PPP.

The present disclosure also provides methods for improving PPP symptoms in a subject based on indices used to measure the disease state.

Treatment of PPP using an antibody disclosed herein may also be determined using measures known in the art. Treatment of PPP may be determined using any of the measures known in the art, e.g., improvement in ppPASI, or any measure known to those in the art.

The ppPASI is an assessment tool based on the Psoriasis Area and Severity Index that is widely used for assessing severity of chronic plaque psoriasis. Parameters including severity, erythema, total number of pustules and desquamation are scored on a scale of 1-4, then corrected for area and site involved (palm or sole). The sum of the four values produces the final ppPASI which ranges between 0 (no PPP) and 72 (the most severe PPP) (Bhushan, et al., Br J Dermatol, 145: 546-53, 2001). ppPASI can be assessed at screening, prior to administration. In one example, administration of an antibody as disclosed herein reduces a ppPASI score. In one example, a subject has a ppPASI score of ≥12 prior to commencing treatment as described herein. In one example, a subject has a ppPASI score of <12 following treatment as described herein.

In one example, administration of an antibody as disclosed herein reduces Palm-Sole Physician Global Assessment (PGA) score in a subject suffering from PPP. The PGA is an average assessment of all psoriatic lesions based on erythema, scale, and induration (Robinson, 2011). PGA can be assessed prior to administration of the antibody.

Other response indicies for PPP include: the PASI scoring system, Investigator's Global Assessment mod 2011 (IGA mod 2011), Dermatology Life Quality Index (DLQI) and Subject's Global Assessment (SGA), Work Productivity and Activity Impairment Questionnaire-Psoriasis (WPAI-PSO), Palmar-Pustular Quality of Life Index (ppQoL-Index).

### Antibodies

Exemplary antibodies antibodies bind to G-CSFR and inhibit G-CSF signaling. Such antibodies are described in WO2012/171057.

Exemplary antibodies bind to G-CSF and inhibit G-CSF signaling. Such antibodies are described in WO2018/145206.

Methods for generating antibodies are known in the art and/or described in Harlow and Lane (editors) Antibodies: A Laboratory Manual, Cold Spring Harbor Laboratory, (1988). Generally, in such methods G-CSFR or G-CSF (e.g., hG-CSFR or hG-CSF) or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567; and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539, or U.S. Pat. No. 5,585,089. Other methods for humanizing an antibody are not excluded. Exemplary humanized antibodies that bind G-CSF and inhibit G-CSF signaling are described in WO2018/145206.

The term “human antibody” as used herein refers to antibodies having variable regions (e.g. V_{H}, V_{L}) and, optionally constant regions derived from or corresponding to sequences found in humans, e.g. in the human germline or somatic cells.

Exemplary human antibodies are described herein and include C1.2 and C1.2G and/or variable regions thereof. These human antibodies provide an advantage of reduced immunogenicity in a human compared to non-human antibodies. Exemplary antibodies are described in WO2012/171057, which is incorporated herein by reference.

Additional Compounds that Inhibit G-CSF Signaling

In one example, the compound that inhibits G-CSF signaling binds to G-CSF or to G-CSFR. In one example, the compound that inhibits G-CSF signaling binds to G-CSF. In one example, the compound that inhibits G-CSF signaling binds to G-CSFR.

In one example, the compound that inhibits G-CSF signaling is a protein.

In one example, the compound that inhibits G-CSF signaling is a protein comprising an antibody variable region that binds to or specifically binds to G-CSFR and neutralizes G-CSF signaling. Reference herein to a protein or antibody that “binds to” G-CSFR provides literal support for a protein or antibody that “binds specifically to” G-CSFR.

In some examples, the compound that inhibits G-CSF signaling is a protein comprising a Fv. In some examples, the protein is selected from the group consisting of:
(i) a single chain Fv fragment (scFv);
(ii) a dimeric scFv (di-scFv); or
(iv) a diabody;
(v) a triabody;
(vi) a tetrabody;
(vii) a Fab;
(viii) a F(ab′)₂;
(ix) a Fv;
(x) one of (i) to (ix) linked to a constant region of an antibody, Fc or a heavy chain constant domain (C_{H}) 2 and/or C_{H}3; or
(xi) one of (i) to (ix) linked to albumin, functional fragments or variants thereof or a protein (e.g., antibody or antigen binding fragment thereof) that binds to albumin;

In one example, a compound that inhibits G-CSF signaling is a protein comprising a Fc region of an antibody.

In one example, the protein is an antibody which binds to hG-CSFR expressed on the surface of a cell at an affinity of at least about 5 nM. In one example, the protein is an antibody which binds to hG-CSFR expressed on the surface of a cell at an affinity of at least about 4 nM. In one example, the protein is an antibody which binds to hG-CSFR expressed on the surface of a cell at an affinity of at least about 3 nM. In one example, the protein is an antibody which binds to hG-CSFR expressed on the surface of a cell at an affinity of at least about 2 nM. In one example, the protein is an antibody which binds to hG-CSFR expressed on the surface of a cell at an affinity of at least about 1 nM.

In one example, the protein is an antibody which inhibits G-CSF-induced proliferation of a BaF3 cell expressing hG-CSFR with an IC50 of at least about 5 nM. In one example, the protein is an antibody which inhibits G-CSF-induced proliferation of a BaF3 cell expressing hG-CSFR with an IC50 of at least about 4 nM. In one example, the protein is an antibody which inhibits G-CSF-induced proliferation of a BaF3 cell expressing hG-CSFR with an IC50 of at least about 3 nM. In one example, the protein is an antibody which inhibits G-CSF-induced proliferation of a BaF3 cell expressing hG-CSFR with an IC50 of at least about 2 nM. In one example, the protein is an antibody which inhibits G-CSF-induced proliferation of a BaF3 cell expressing hG-CSFR with an IC50 of at least about 1 nM. In one example, the protein is an antibody which inhibits G-CSF-induced proliferation of a BaF3 cell expressing hG-CSFR with an IC50 of at least about 0.5 nM.

### Single-Domain Antibodies

In some examples, a compound of the disclosure is a protein that is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, Mass.; see, e.g., U.S. Pat. No. 6,248,516).

### Diabodies, Triabodies, Tetrabodies

In some examples, a protein of the disclosure is or comprises a diabody, triabody, tetrabody or higher order protein complex such as those described in WO98/044001 and/or WO94/007921.

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Antibody Fragments

The present disclosure also contemplates other antibodies and antibody fragments, such as:

(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see WO2002088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Soluble G-CSFR

The present disclosure also contemplates a soluble form of the G-CSFR which competes with the naturally occurring membrane-associated G-CSFR for G-CSF interaction. Those skilled in the art can readily prepare soluble forms of the receptor, see for example U.S. Pat. No. 5,589,456 and Honjo et al, Acta Crystallograph Sect F Struct Biol Cryst Commun. 61(Pt 8):788-790, 2005.

### Constant Regions

Sequences of constant regions useful in compounds and antibodies of the present disclosure may be obtained from a number of different sources. In some examples, the constant region or portion thereof of the antibody is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Protein Production

In one example, an antibody described herein according to any example is recombinant.

In the case of a recombinant antibody, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the antibody. Exemplary cells used for expressing an antibody are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding an antibody (e.g., derived from the information provided herein), an enhancer element, a promoter, and a transcription termination sequence. The skilled artisan will be aware of suitable sequences for expression of an antibody. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The host cells used to produce the antibody may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating an antibody are known in the art and/or described herein. Where an antibody is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the antibody, e.g., using continuous centrifugation.

The antibody prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

### Assaying Activity of an Antibody

### Binding to G-CSFR and Mutants Thereof

It will be apparent to the skilled artisan from the disclosure herein that compounds or antibodies of the present disclosure bind to the ligand binding domain of hG-CSFR and to specific mutant forms of the ligand binding domain of hG-CSFR (e.g., SEQ ID NO: 1 without or with certain point mutations) and/or bind to both human and cynomolgus monkey G-CSFR. Methods for assessing binding to a compound or an antibody are known in the art, e.g., as described in Scopes (In: Protein purification: principles and practice, Third Edition, Springer Verlag, 1994). Such a method generally involves labeling the compound or antibody and contacting it with immobilized G-CSFR. Following washing to remove non-specific bound compound or antibody, the amount of label and, as a consequence, bound compound or antibody is detected. Of course, the compound or antibody can be immobilized and G-CSFR signaling labeled. Panning-type assays can also be used. Alternatively, or additionally, surface plasmon resonance assays can be used.

In one example, a compound or an antibody of the present disclosure binds to a polypeptide of SEQ ID NO: 1 in which an alanine is substituted for the lysine at position 167 of SEQ ID NO: 1 and/or in which an alanine is substituted for the histidine at position 168 of SEQ ID NO: 1 at substantially the same level (e.g., within 10% or 5% or 1%) as it binds to SEQ ID NO: 1.

In one example, a compound or an antibody of the present disclosure binds to a polypeptide of SEQ ID NO: 1 in which an alanine is substituted for the arginine at position 287 of SEQ ID NO: 1 at a level at least about 100 fold or 150 fold or 160 fold or 200 fold lower than it binds to a polypeptide of SEQ ID NO: 1. In one example, a compound or an antibody of the present disclosure binds to a polypeptide of SEQ ID NO: 1 in which an alanine is substituted for the arginine at position 287 of SEQ ID NO: 1 at a level at least about 160 fold lower than it binds to a polypeptide of SEQ ID NO: 1.

In one example, a compound or an antibody of the present disclosure binds to a polypeptide of SEQ ID NO: 1 in which an alanine is substituted for the histidine at position 237 of SEQ ID NO: 1 at a level at least about 20 fold or 40 fold or 50 fold or 60 fold lower than it binds to a polypeptide of SEQ ID NO: 1. In one example, a compound or an antibody of the present disclosure binds to a polypeptide of SEQ ID NO: 1 in which an alanine is substituted for the histidine at position 237 of SEQ ID NO: 1 at a level at least about 50 fold lower than it binds to a polypeptide of SEQ ID NO: 1.

In one example, a compound or an antibody of the present disclosure binds to a polypeptide of SEQ ID NO: 1 in which an alanine is substituted for the methionine at position 198 of SEQ ID NO: 1 at a level at least about 20 fold or 40 fold or 60 fold or 70 fold lower than it binds to a polypeptide of SEQ ID NO: 1. In one example, a compound or an antibody of the present disclosure binds to a polypeptide of SEQ ID NO: 1 in which an alanine is substituted for the methionine at position 198 of SEQ ID NO: 1 at a level at least about 40 fold lower than it binds to a polypeptide of SEQ ID NO: 1.

In one example, a compound or an antibody of the present disclosure binds to a polypeptide of SEQ ID NO: 1 in which an alanine is substituted for the tyrosine at position 172 of SEQ ID NO: 1 at a level at least about 20 fold or 30 fold or 40 fold lower than it binds to a polypeptide of SEQ ID NO: 1. In one example, a compound or an antibody of the present disclosure binds to a polypeptide of SEQ ID NO: 1 in which an alanine is substituted for the tyrosine at position 172 of SEQ ID NO: 1 at a level at least about 40 fold lower than it binds to a polypeptide of SEQ ID NO: 1.

In one example, a compound or an antibody of the present disclosure binds to a polypeptide of SEQ ID NO: 1 in which an alanine is substituted for the leucine at position 171 of SEQ ID NO: 1 at a level at least about 100 fold or 120 fold or 130 fold or 140 fold lower than it binds to a polypeptide of SEQ ID NO: 1. In one example, a compound or an antibody of the present disclosure binds to a polypeptide of SEQ ID NO: 1 in which an alanine is substituted for the leucine at position 171 of SEQ ID NO: 1 at a level at least about 140 fold lower than it binds to a polypeptide of SEQ ID NO: 1.

In one example, a compound or an antibody of the present disclosure binds to a polypeptide of SEQ ID NO: 1 in which an alanine is substituted for the leucine at a position 111 of SEQ ID NO: 1 at a level at least about 20 fold or 40 fold or 60 fold or 70 fold lower than it binds to a polypeptide of SEQ ID NO: 1. In one example, a compound or an antibody of the present disclosure binds to a polypeptide of SEQ ID NO: 1 in which an alanine is substituted for the leucine at a position 111 of SEQ ID NO: 1 at a level at least about 60 fold lower than it binds to a polypeptide of SEQ ID NO: 1.

In one example, a compound or an antibody of the present disclosure binds to a polypeptide of SEQ ID NO: 1 in which an alanine is substituted for the histidine at position 168 of SEQ ID NO: 1 at a level no more than 5 fold or 4 fold or 3 fold or 2 fold or 1 fold lower than it binds to a polypeptide of SEQ ID NO: 1.

In one example, a compound or an antibody of the present disclosure binds to a polypeptide of SEQ ID NO: 1 in which an alanine is substituted for the lysine at position 167 of SEQ ID NO: 1 at a level no more than 5 fold or 4 fold or 3 fold or 2 fold or 1 fold lower than it binds to a polypeptide of SEQ ID NO: 1.

The level of binding is conveniently determined using a biosensor.

The present disclosure contemplates any combination of the foregoing characteristics. In one example, an antibody described herein has all of the binding characteristics set forth in the preceding seven paragraphs.

### Epitope Mapping

In another example, the epitope bound by a compound or an antibody described herein is mapped. Epitope mapping methods will be apparent to the skilled artisan. For example, a series of overlapping peptides spanning the hG-CSFR sequence or a region thereof comprising an epitope of interest, e.g., peptides comprising 10-15 amino acids are produced. The compound or antibody is then contacted to each peptide and the peptide(s) to which it binds determined. This permits determination of peptide(s) comprising the epitope to which the antibody binds. If multiple non-contiguous peptides are bound by the protein, the antibody may bind a conformational epitope.

Alternatively, or in addition, amino acid residues within hG-CSFR are mutated, e.g., by alanine scanning mutagenesis, and mutations that reduce or prevent protein binding are determined. Any mutation that reduces or prevents binding of the compound or antibody is likely to be within the epitope bound by the protein.

A further method is exemplified herein, and involves binding hG-CSFR or a region thereof to an immobilized compound or antibody of the present disclosure and digesting the resulting complex with proteases. Peptide that remains bound to the immobilized protein are then isolated and analyzed, e.g., using mass spectrometry, to determine their sequence.

### Determining Competitive Binding

Assays for determining an compound or antibody that competitively inhibits binding of monoclonal antibody C1.2 or C1.2G will be apparent to the skilled artisan. For example, C1.2 or C1.2G is conjugated to a detectable label, e.g., a fluorescent label or a radioactive label. The labeled antibody and the test compound or antibody are then mixed and contacted with hG-CSFR or a region thereof (e.g., a polypeptide comprising SEQ ID NO: 1) or a cell expressing same. The level of labeled C1.2 or C1.2G is then determined and compared to the level determined when the labeled compound or antibody is contacted with the hG-CSFR, region or cells in the absence of the test antibody. If the level of labeled C1.2 or C1.2G is reduced in the presence of the test compound or antibody compared to the absence of the antibody, the antibody is considered to competitively inhibit binding of C1.2 or C1.2G to hG-CSFR.

Optionally, the test compound or antibody is conjugated to different label to C1.2 or C1.2G. This alternate labeling permits detection of the level of binding of the test antibody to hG-CSFR or the region thereof or the cell.

In another example, the compound or antibody is permitted to bind to hG-CSFR or a region thereof (e.g., a polypeptide comprising SEQ ID NO: 1) or a cell expressing same prior to contacting the hG-CSFR, region or cell with C1.2 or C1.2G. A reduction in the amount of bound C1.2 or C1.2G in the presence of the compound or antibody compared to in the absence of the compound or antibody indicates that the protein competitively inhibits C1.2 or C1.2G binding to hG-CSFR. A reciprocal assay can also be performed using labeled compound or antibody and first allowing C1.2 or C1.2G to bind to G-CSFR. In this case, a reduced amount of labeled compound or antibody bound to hG-CSFR in the presence of C1.2 or C1.2G compared to in the absence of C1.2 or C1.2G indicates that the compound or antibody competitively inhibits binding of C1.2 or C1.2G to hG-CSFR.

Any of the foregoing assays can be performed with a mutant form of hG-CSFR and/or SEQ ID NO: 1 and/or a ligand binding region of hG-CSFR to which C1.2 or C1.2G binds, e.g., as described herein.

### Determining Neutralization

In some examples of the present disclosure, a compound or an antibody is capable of neutralizing hG-CSFR signaling.

Various assays are known in the art for assessing the ability of a compound or an antibody to neutralize signaling of a ligand through a receptor.

In one example, the compound or antibody that inhibits G-CSF signaling reduces or prevents G-CSF binding to the hG-CSFR. These assays can be performed as a competitive binding assay as described herein using labeled G-CSF and/or labeled protein.

In another example, the compound or antibody that inhibits G-CSF signaling reduces formation of CFU-G when CD34⁺ bone marrow cells are cultured in the presence of G-CSF. In such assays, CD34⁺ bone marrow cells are cultured in a semi-solid cell culture medium in the presence of G-CSF (e.g., about 10 ng/ml cell culture medium) and, optionally stem cell factor (e.g., about 10 ng/ml cell culture medium) in the presence or absence of a test compound. After a sufficient time for granulocyte clones (CFU-G) to form, the number of clones or colonies is determined. A reduction in the number of colonies in the presence of the antibody that inhibits G-CSF signaling compared to in the absence of the compound or antibody that inhibits G-CSF signaling indicates that the compound or antibody that inhibits G-CSF signaling neutralizes G-CSF signaling. By testing multiple concentrations of the antibody that inhibits G-CSF signaling an IC₅₀ is determined, i.e., a concentration at which 50% of the maximum inhibition of CFU-G formation occurs. In one example, the IC₅₀ is 0.2 nM or less, such as 0.1 nM or less, for example, 0.09 nM or less, or 0.08 nM or less, or 0.07 nM or less, or 0.06 nM or less or 0.05 nM or less. In one example, the IC₅₀ is 0.04 nM or less. In another example, the IC₅₀ is 0.02 nM or less. The foregoing IC₅₀s relate to any CFU-G assay described herein.

In a further example, the compound or antibody that inhibits G-CSF signaling reduces proliferation of cells (e.g., BaF3 cells) expressing hG-CSFR which are cultured in the presence of G-CSF. Cells are cultured in the presence of G-CSF (e.g., 0.5 ng/ml) and the presence or absence of a test compound or antibody. Methods for assessing cell proliferation are known in the art and include, for example, MTT reduction and thymidine incorporation. A compound or antibody that reduces the level of proliferation compared to the level observed in the absence of the compound or antibody is considered to neutralize G-CSF signaling. By testing multiple concentrations of the compound or antibody an IC₅₀ is determined, i.e., a concentration at which 50% of the maximum inhibition of cell proliferation occurs. In one example, the IC₅₀ is 6 nM or less, such as 5.9 nM or less. In another example, the IC₅₀ s 2 nM or less or 1 nM or less or 0.7 nM or cell or 0.6 nM or less or 0.5 nM or less. The foregoing IC₅₀s relate to any cell proliferation assay described herein.

In a further example, the compound or antibody that inhibits G-CSF signaling reduces mobilization of hematopoietic stem cells and/or endothelial progenitor cells in vivo following G-CSF administration and/or reduces the number of neutrophils in vivo, e.g., following G-CSF administration (however this is not essential). For example, the compound or antibody that inhibits G-CSF signaling is administered, optionally before, at the time of or after administration of G-CSF or a modified form thereof (e.g., PEGylated G-CSF or filgrastim). The number of hematopoietic stem cells (e.g., expressing CD34 and/or Thy1) and/or endothelial progenitor cells (e.g., expressing CD34 and VEGFR2) and/or neutrophils (identified morphologically and/or expressing e.g., CD10, CD14, CD31 and/or CD88) is assessed. A compound or antibody that reduces the level of the cell(s) compared to the level observed in the absence of the antibody is considered to neutralize G-CSF signaling. In one example, the compound or antibody that inhibits G-CSF signaling reduces the number of neutrophils without inducing neutropenia.

Other methods for assessing neutralization of G-CSF signaling are contemplated by the present disclosure.

### Compositions

Compositions of a compound or an antibody of the present disclosure is/are administered intravenously or subcutaneously. In one example, the antibody/composition is administered intravenously.

Methods for preparing a compound or antibody into a suitable form for administration (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or subcutaneous administration. The compositions for administration will commonly comprise a solution of the antibody dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of compound of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution. Nonaqueous vehicles such as additives that enhance isotonicity and chemical stability, e.g., buffers and preservatives may be used.

### Combination Therapies

In one example, a compound or antibody of the present disclosure is administered in combination with another compound useful for treating a disease or condition described herein, either as combined or additional treatment steps or as additional components of a therapeutic formulation.

For example, the other compound is an anti-inflammatory compound, e.g., methotrexate or a non-steroidal anti-inflammatory compound. Alternatively, or additionally, the other compound is an immunosuppressant. Alternatively, or additionally, the other compound is a corticosteroid, such as prednisone and/or prednisolone. In on example, the other compound is methotrexate. Alternatively, or additionally, the other compound is cyclophosphamide.

In one example, the compound or antibody is administered simultaneously with the other compound. In one example, the antibody that inhibits G-CSF signaling is administered before the other compound. In one example, the antibody that inhibits G-CSF signaling is administered after the other compound.

In some examples, the compound or antibody is administered in combination with a cell. In some examples, the cell is a stem cell, such as a mesenchymal stem cell.

In some examples, the compound or antibody is administered in combination with a gene therapy.

In some examples, the compound or antibody is administered in combination with a non-pharmaceutical intervention, for example, apharesis, such as plasmapheresis, cytapheresis, leukapheresis, granulocyte and/or monocyte apheresis. In this context, the compound or antibody can be administered during the period of time in which the non-pharmaceutical intervention is being performed and will be considered “in combination with” the non-pharmaceutical intervention. For example, the non-pharmaceutical intervention may be granulocyte and/or monocyte apheresis, which is performed once per week for five weeks and the antibody or compound can be administered over this time period. In one example, the antibody or compound is administered before the non-pharmaceutical intervention. In one example, the antibody or compound is administered after the non-pharmaceutical intervention.

Another non-pharmaceutical intervention is light therapy. Light therapy is used to treat some neutrophilic dermatoses.

### Dosing

In one example, the compound or antibody is administered at a dose of between 0.1 mg/kg and 1 mg/kg. For example, the compound or antibody is administered at a dose of between 0.1 mg/kg and 0.9 mg/kg.

In one example, the compound or antibody is administered at a dose of between 0.1 mg/kg and 0.8 mg/kg.

In one example, the compound or antibody is administered at a dose of between 0.1 mg/kg and 0.6 mg/kg.

In one example, the compound or antibody is administered at a dose of between 0.3 mg/kg and 0.6 mg/kg.

In one example, the compound or antibody is administered at a dose of between 0.1 mg/kg and 0.3 mg/kg.

In one example, the compound or antibody is administered at a dose of about 0.1 mg/kg. In one example, the compound or antibody is administered at a dose of 0.1 mg/kg.

In one example, the compound or antibody is administered at a dose of about 0.3 mg/kg. In one example, the compound or antibody is administered at a dose of 0.3 mg/kg.

In one example, the compound or antibody is administered at a dose of about 0.6 mg/kg. In one example, the compound or antibody is administered at a dose of 0.6 mg/kg.

In one example, the compound or antibody is administered multiple times. For example, the compound or antibody is administered once every 5 to 40 days. In some examples, the compound or antibody is not administered on consecutive days or within the same week.

For example, the compound or antibody is administered every 14 to 28 days. For example, the compound or antibody is administered every 20 to 25 days.

For example, the compound is administered every 7 days or 8 days or 9 days or 10 days or 11 days or 12 days or 13 days or 14 days or 15 days or 16 days or 17 days or 18 days or 19 days or 20 days or 21 days or 22 days or 23 days or 24 days or 25 days or 26 days or 27 days or 28 days.

In one example, the compound is administered biweekly or triweekly or every 4 weeks.

For example, the compound or antibody is administered multiple times, wherein the compound or antibody is administered once every 21 days. In this regard “every 21 days” (or any other number) will be understood by the skilled person to mean that the subsequent administration is performed on the 21^{st} day following the immediately prior administration.

In one example, a method of the disclosure comprises administering an antibody that binds to or specifically binds to granulocyte-colony stimulating factor receptor (G-CSFR), wherein the antibody is administered multiple times once every 21 days and wherein the antibody comprises:

(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

In one example, a method of the disclosure comprises administering a composition comprising an antibody that binds to or specifically binds to granulocyte-colony stimulating factor receptor (G-CSFR), wherein the antibody is administered multiple times once every 21 days and wherein the composition comprises at least two or all three of the following:

(i) an antibody comprising a heavy chain comprising a sequence set forth in SEQ ID NO: 14 and a light chain comprising a sequence set forth in SEQ ID NO: 15;
(ii) an antibody comprising a heavy chain comprising a sequence set forth in SEQ ID NO: 16 and a light chain comprising a sequence set forth in SEQ ID NO: 15; and/or
(iii) an antibody comprising a heavy chain comprising a sequence set forth in SEQ ID NO: 16 and a heavy chain comprising a sequence set forth in SEQ ID NO: 14 and two light chains comprising a sequence set forth in SEQ ID NO: 15.

In one example, the compound or antibody is administered for a set period or number of doses. For example, the compound or antibody is administered for 1 month or 3 months or 6 months or 12 months. In another example, five or 10 or 15 or 20 doses of the antibody or compound is administered.

In another example, the compound or antibody is administered chronically or an on ongoing basis, e.g., for months or years and the present disclosure is not limited to a specific time period unless stated otherwise.

In one example, the compound or antibody is administered until the condition or symptoms of the condition or resolved or managed. For example, in the case of an “active” form of a condition, the compound or antibody is administered until the condition is no longer considered active.

In the case of HS or PPP, the compound or antibody is administered until the subject no longer has any visible lesions or pustules.

In one example, the compound or antibody is administered to induce remission of a condition. In another example, the compound is administered to maintain remission of a condition.

In one example, one or more loading doses of the compound is administered followed by one or more maintenance doses. Generally, the loading doses will be higher or administered with a shorter time period between them than the maintenance doses.

For example, one or two or three or more loading doses of the antibody or compound is administered to the subject, e.g., to induce remission, followed by ongoing maintenance doses. These maintenance doses may continue indefinitely or until the subject suffers an adverse reaction or until the condition returns or worsens upon which one or more loading doses may be required.

In some examples, the loading dose is 1.5 times or two times or three times higher than the maintenance dose. As an example, the loading lose can be 0.9 mg/kg and the maintenance dose can be 0.3 mg/kg or the loading dose can be 0.3 mg/kg and the maintenance dose can be 0.1 mg/kg or the loading dose can be 0.6 mg/kg and the maintenance dose can be 0.1 mg/kg.

In some examples, the loading dose is administered more frequently than the maintenance dose. For example, the loading dose is administered weekly or biweekly and the maintenance dose is administered every 21 days. In this case, the dosages of the loading and maintenance dose can be the same or different.

In the case of a subject that is not adequately responding to treatment, more frequent or higher doses may be administered.

In another example, for subjects experiencing an adverse reaction, a dose may be split over numerous days in one week or over numerous consecutive days.

In one example, for subjects experiencing neutropenia as an adverse reaction, a caregiver may be instructed to cease treatment. For example, if the subject experiences neutropenia for more than 2 consecutive days or 3 consecutive days, treatment may be ceased.

In one example, for subjects experiencing neutropenia as an adverse reaction, a caregiver may be instructed to skip the next dose. For example, if the subject experiences neutropenia for more than 2 consecutive days or 3 consecutive days, the next dose may be skipped.

Optionally, the subject suffering from neutropenia may be treated with G-CSF or GM-CSF to treat the neutropenia.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Example 1—Safety, Pharmacokinetics (PK) and Pharmacodynamics (PD) of C1.2G, an Antibody that Binds to G-CSFR, Administered to Healthy Adult Subjects

A Phase 1 clinical trial was conducted to assess the safety and tolerability of single ascending dose (Parts A and B) and repeated (Part C) intravenous (IV) infusions of CSL324 (also referred to as C1.2G herein) in healthy subjects.

### Method

The trial was a first-in-human, single center, randomized, double-blind, placebo-controlled study assessing the safety, tolerability, PK, and pharmacodynamics (PD) of single ascending doses and repeat doses of IV CSL324 in healthy human subjects. The study consisted of 3 parts: Parts A, B, and C. Regular blinded review of safety, tolerability, PK, and selected PD data was conducted by the Safety Review Committee (SRC) to guide dose selection. This trial is described at the Australian New Zealand Clinical Trials Registry (ANZCTR) under Registration Number ACTRN12616000846426, title: “Dose escalation, placebo-controlled phase 1 study to assess the safety and tolerability of CSL324 in healthy adults”.

### Part A: Single Ascending Dose

Part A assessed single ascending doses of CSL324 administered to 5 sequential cohorts (Cohorts Al to A5). Each cohort comprised 6 subjects randomized to receive either CSL324 (n=4) or placebo (n=2) on Day 1. Single ascending doses of 0.1, 0.3, 1.0, 3.0, and 10 mg/kg of CSL324 were planned for the 5 sequential cohorts. At the recommendation of the SRC, the highest dose administered was 1.0 mg/kg of CSL324 (Cohort A3); Cohorts A4 and A5 received intermediate doses of 0.6 and 0.8 mg/kg CSL324, respectively. Subjects were followed up until Day 85.

Part B—Single Ascending Dose with G-CSF Challenge

Part B assessed single doses of CSL324 during a G-CSF challenge. Cohorts B1, B2, and B3 each comprised 4 subjects randomized to receive either CSL324 (n=3) or placebo (n=1) on Day 1. Cohort B1 received 0.1 mg/kg CSL324, and Cohorts B2 and B3 received 0.3 and 0.8 mg/kg CSL324, respectively, at the recommendation of the SRC. Subjects were administered a G-CSF challenge (5 μg/kg filgrastim) before and after CSL324 (on Days −3, −2, −1, 1, 2, and 3). Cohort B4 comprised 6 subjects randomized to receive either CSL324 (n=4) or placebo (n=2) on Day 1. Subjects received 0.8 mg/kg CSL324 and were administered a G-CSF challenge (5 μg/kg filgrastim) after CSL324 only (on Days 2, 3, and 4). Subjects were followed up until Day 85.

### Part C—Repeat Dose

Part C assessed 3 repeat doses of CSL324 administered at 21-day intervals (Days 1, 22, and 43). Ten subjects were randomized to receive either CSL324 (n=6) or placebo (n=4). Subjects were administered 0.6 mg/kg CSL324 at the recommendation of the SRC. Subjects were followed up until Day 126.

### Safety, Pharmacokinetic (PK), and Pharmacodynamic (PD) Assessments

In all study parts, safety assessments included adverse events (AEs), vital signs including orthostatic challenge, physical and neurological examination, 12-lead electrocardiogram (ECG), cardiac monitoring using ECG telemetry, clinical laboratory tests (hematology, blood chemistry, coagulation, and urinalysis), fatigue measured on a visual analogue scale, and CSL324 immunogenicity.

CSL324 was measured in serum (all cohorts) and cerebrospinal fluid (Cohort A5 only).

PD assessments included neutrophil functional attributes (phagocytic activity, oxidative burst activity, G-CSF receptor phospho-signal transducer and activator of transcription-3 [pSTAT-3] signaling [Part A only], and granulocyte macrophage colony stimulating factor [GM-CSF] receptor pSTAT-3 signaling [Parts A and C only]); neutrophil G-CSF receptor occupancy/saturation (Parts A and C only); and serum G-CSF, cytokines, and chemokines.

### Diagnosis and Main Criteria for Inclusion

Healthy male or female subjects, 18 to 55 years of age, with body mass index of 18.5 to 32.0 kg/m2 (inclusive) and weight >50 kg and <100 kg, who provided written informed consent. Female subjects were to be of non-childbearing potential; male subjects and their female spouse/partner of childbearing potential were to use 2 forms of highly effective birth control from Screening until 90 days after the final IV infusion.

Subjects were excluded if they had a history or evidence of any clinically significant cardiovascular, gastrointestinal, endocrine, hematologic, hepatic, immunologic, metabolic, urologic, pulmonary, neurologic, dermatologic, psychiatric, renal and/or other major disease or malignancy, as judged by the Investigator; a history of venous thrombosis, polycythemia, or thrombophilia; a history of autoimmune disease; cyclic neutropenia or a Screening absolute neutrophil count (ANC)<2.0×10⁹/L; any clinically significant abnormality identified at Screening or site admission; pulse rate <40 or >100 beats per minute, mean systolic blood pressure >145 mmHg, or mean diastolic blood pressure >90 mmHg at Screening or site admission; mean corrected QT interval using Fridericia's formula >450 msec at Screening; or use of any prescribed or non-prescribed drugs in the 10 days before IV infusion, except for the occasional use of paracetamol (up to 2 g/day). For Parts A and B only, subjects were excluded if they had any tattoo or compromised skin health, or a history of keloid formation, hypertrophic scarring, or lymphangitis.

### CSL324 Antibody Dose and Mode of Administration

CSL324 was provided as a sterile solution for injection in 10 mL vials. CSL324 was administered IV at a volume determined by the subject's weight on Day 1 and cohort dose.

Placebo, 0.9% sodium chloride, was administered IV at an equivalent volume to CSL324 according to the subject's weight on Day 1 and cohort dose.

All CSL324 infusions were to be given over 60±5 minutes in a forearm vein using a syringe pump (doses <1.0 mg/kg).

### Duration of Treatment

Subjects in Parts A or B received CSL324 or placebo as a single dose on Day 1, and were followed up until Day 85. Subjects in Part C received 3 doses of CSL324 or placebo at 21-day intervals on Days 1, 22, and 43, and were followed up until Day 126.

### Criteria for Evaluation:

Primary endpoint: Incidence, causality, and severity of AEs during the study.

### Secondary Endpoints:

Pharmacokinetic parameters of CSL324 in serum:
Parts A and B:
AUC_{0-inf}—Area under the concentration-time curve from time 0 extrapolated to time infinity
AUC₀₋ₜ—Area under the concentration-time curve from time 0 to collection time t
Cₘₐₓ—Maximum concentration
CLₜₒₜ—Total systemic clearance after IV dosing
tₘₐₓ—Time of maximum concentration
t_{1/2}—Terminal elimination half-life
V_{z}—Volume of distribution after IV dosing during the terminal elimination phase
Part C:
AUC₀₋ₜ—Area under the concentration-time curve from time 0 to collection time t
AUC₀₋ₜₐᵤ—Area under the concentration-time curve during dosing interval at steady state
C_{min,ss} Minimum (trough) concentration at steady state
C_{max,ss}—Maximum concentration at steady state
t_{max,ss}—Time of maximum concentration at steady state
t_{1/2,ss}—Terminal elimination half-life at steady state
CL_{tot,ss}—Total systemic clearance at steady state after IV dosing
V_{z,ss}—Volume of distribution at steady state after IV dosing during the terminal elimination phase
Concentrations of CSL324 and of G-CSF in cerebrospinal fluid (Cohort A5 only).
Presence of anti-CSL324 antibodies in serum.
Non-compartmental PD parameters for ANC, including the maximum effect (Eₘₐₓ) of ANC from Day 1 and the area under the effect curve from time 0 to 24 hours for ANC (AUEC_{0-24,ANC}), after G-CSF challenge following CSL324 or placebo dosing (Part B only).

### Statistical Methods

### Analysis Populations

The Full Analysis Set (FAS) comprised all subjects who provided written informed consent and who were eligible for inclusion in the study after Screening. The FAS was used for demographics, baseline characteristics, and immunogenicity. The Safety Population comprised all subjects who received at least 1 dose of CSL324, analyzed according to the dose and medication received, and was used for all safety analyses.

The PK Population comprised all subjects who received at least 1 dose of CSL324 and had at least 1 measured PK concentration, and was used for all PK analyses.

The PD Population comprised all subjects who received at least 1 dose of CSL324 and for whom PD data were available before CSL324 infusion and for at least 1 time point after CSL324 infusion. The PD Population was used for all PD analyses.

### General Considerations

All data were listed by subject. Summary statistics were presented using descriptive statistics. All statistical tests were 2-sided and performed at the 5% level of significance, unless otherwise stated.

### Pharmacokinetic (PK) Analyses

PK parameters were derived from serum CSL324 concentrations by standard noncompartmental analysis using actual sampling times. Dose proportionality was assessed for the PK parameters Cmax, AUC₀₋ₜ, and AUC_{0-inf} for the single dose cohorts in Part A and Part B separately. Dose proportionality was analyzed using a power model which included loge-transformed body weight-adjusted dose level as an independent variable. Linear proportionality between the PK parameter and dose could be declared if the 90% confidence interval (CI) was within the critical interval of 0.85 to 1.15. Correlation of PK parameters Cₘₐₓ, AUC₀₋ₜ, and AUC_{0-inf} with total dose (mg) and body weight-adjusted dose (mg/kg) was investigated for Part A and Part B using Pearson correlation analysis.

The relative bioavailability of CSL324 without (Part A) and with (Part B) co-administration of G-CSF was assessed using a mixed-effect model (with treatment as fixed effect and subject as random effect) and the loge-transformed PK parameters Cmax, AUC₀₋ₜ, and AUC_{0-inf}. Administration of CSL324 without (Part A) and with (Part B) co-administration of G-CSF was considered equivalent if the 90% CI for the geometric mean ratio was between 80% and 125% for any comparison.

Attainment of steady state after 3 doses of CSL324 every 21 days (Part C) was assessed by repeated measures analysis of variance (ANOVA) of minimum trough concentration (Cmin). The first non-significant comparison was the dosing interval at which steady state was attained.

### Pharmacodynamic (PD) Analyses

PD parameters were derived using standard noncompartmental analysis. The PD parameters for serum cytokine and chemokine concentrations, neutrophil phagocytic and oxidative burst activity, G-CSF receptor occupancy, and pSTAT-3 signaling for Part A were compared between each CSL324 dose in Part A and the pooled placebo group for Part A by ANOVA.

Correlation of PD parameters with CSL324 total dose (mg) and body weight-adjusted dose (mg/kg) was investigated using Pearson correlation analysis.

### Safety Analyses

Treatment-emergent AEs (TEAEs) were coded using Medical Dictionary for Regulatory Activities (MedDRA; Version 20.1). The severity of each TEAE was assessed by the Investigator using the National Cancer Institute Common Terminology Criteria for Adverse Events Version 4, except for TEAEs of abnormal ANC values which were graded using Club Phase 1 criteria. Box plot comparisons between subjects with cumulative positive and negative immunogenicity results were done for CSL324 clearance (CLtot or CLtot,ss) and selected PD parameters (ANC and G-CSF concentration).

### Results

### Subject Disposition

A total of 58 subjects provided informed consent and were randomized into the study. In Part A (n=30), 4 subjects received CSL324 and 2 subjects received placebo in each of the 5 cohorts (Cohorts Al to A5). In Part B (n=18), 3 subjects received CSL324 and 1 subject received placebo in each of Cohorts B1 to B3, and 4 subjects received CSL324 and 2 subjects received placebo in Cohort B4. In Part C (n=10), 6 subjects received CSL324 and 4 subjects received placebo.

Overall, 55 subjects (94.8%) completed the study; 1 placebo-treated subject was discontinued from Part A (Cohort A5) due to withdrawn consent, and 2 subjects (1 CSL324-treated and 1 placebo-treated) were discontinued after completing the 3 doses in Part C due to other reasons. Two subjects who completed Part C of the study did not receive CSL324 Dose 3 at the recommendation of the SRC.

### Demographics

Study subjects were male (100%) and predominantly White (65.5%), with a mean age of 30.3 years (range: 19 to 54 years). There were no major differences in demographic characteristics between subjects in Parts A, B, or C. Overall, the subject medical and surgical histories were consistent with a healthy volunteer population.

### Pharmacokinetics (PK)

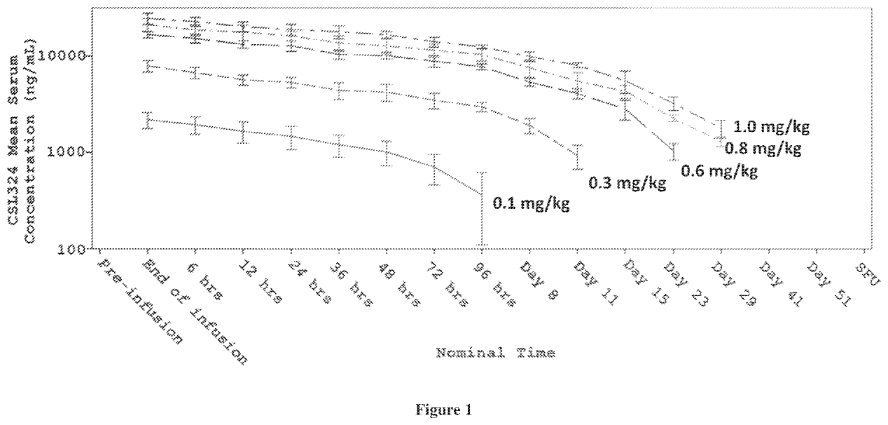

After single IV doses of CSL324, mean serum CSL324 concentrations peaked at the end of infusion, with Cₘₐₓ showing linear proportionality to CSL324 dose (FIG. 1). Exposure to CSL324, measured as AUC₀₋ₜ and AUC_{0-inf}, increased with higher CSL324 doses but did not demonstrate dose linearity as the confidence limits for both parameters were outside the 0.85 to 1.15 critical interval (estimated slope 1.68 [90% CI: 1.58 to 1.79] for AUC₀₋ₜ and 1.67 [90% CI: 1.56 to 1.78] for AUC_{0-inf}). Mean CLₜₒₜ of CSL324 was not constant across the range of doses tested, decreasing by 80% with a 10-fold increase in CSL324 dose.

After single IV doses, mean t_{1/2} ranged from 40.5 hours with 0.1 mg/kg CSL324 to 206 hours with 1.0 mg/kg CSL324. After 3 doses of 0.6 mg/kg CSL324, administered at 21-day intervals, mean t_{1/2} was 251 hours.

Administration of G-CSF before and after CSL324 infusion lowered the relative bioavailability of single CSL324 doses, measured as AUC_{0-inf} and AUC₀₋ₜ, and had minimal effect on Cₘₐₓ. The reduction in CSL324 exposure by G-CSF was greater when G-CSF was administered before and after CSL324 dosing compared with after CSL324 dosing only.

Steady state was not achieved after 3 doses of 0.6 mg/kg CSL324 administered at 21-day intervals based on trough concentrations. Peak mean serum CSL324 concentrations were similar after Dose 1 and Dose 3.

CSL324 was not detectable in cerebrospinal fluid after a single 0.8 mg/kg CSL324 dose.

### Pharmacodynamics

Mean ANC decreased after single and repeat CSL324 doses, administered without G-CSF challenge, when compared with placebo. Across the single CSL324 doses, mean ANC minimum effect (Emir) was lowest with the 1.0 mg/kg CSL324 dose (1.3×10⁹/L) and highest with placebo (2.49×10⁹/L). Mean ANC Eₘᵢₙ decreased to 1×10⁹/L after repeat CSL324 dosing, with Eₘᵢₙ occurring after Dose 3 (at approximately Day 48).

Higher doses of CSL324 (0.3 and 0.8 mg/kg) inhibited the G-CSF-mediated stimulation of elevated ANC; the ANC response to G-CSF challenge was similar with 0.1 mg/kg CSL324 and placebo. ANCs were negatively correlated with CSL324 dose and CSL324 exposure, based on AUC₀₋ₜ.

CSL324 had no apparent effects on neutrophil function when measured ex vivo as neutrophil phagocytic and oxidative burst activity. Higher single doses of CSL324 (0.3 to 1.0 mg/kg) increased the G-CSF half-maximal effective concentration (EC50) for ex vivo stimulation of neutrophil pSTAT-3 signaling compared with placebo; however, the assay data showed large variability, limiting interpretation. No consistent effect of CSL324 was seen on the ratio of GM-CSF stimulated versus unstimulated neutrophil pSTAT-3 signaling.

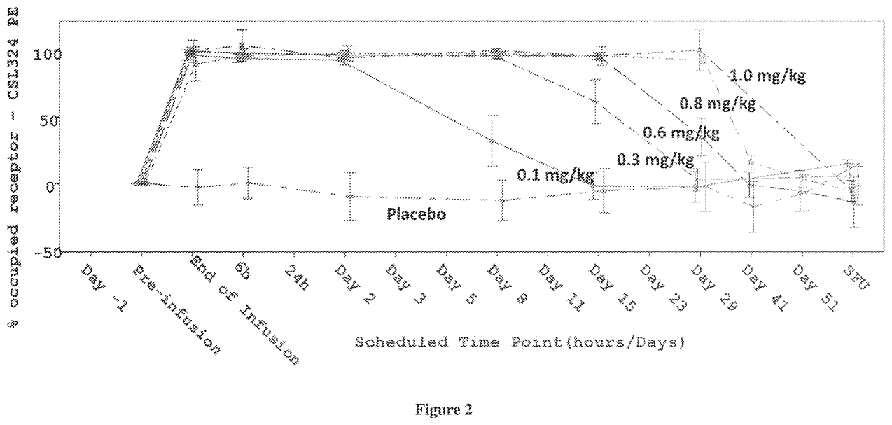

Neutrophil G-CSF receptor saturation was achieved rapidly with single CSL324 doses from 0.1 to 1.0 mg/kg. The duration of approximately 100% receptor occupancy increased with increasing CSL324 dose, lasting until Day 3 with 0.1 mg/kg CSL324 and until Day 29 with 0.8 and 1.0 mg/kg CSL324 (FIG. 2).

Single CSL324 doses increased peak serum G-CSF concentrations and exposure compared with placebo, with G-CSF AUEC₀₋ₜ and AUEC₀₋₂₄ showing a positive correlation with CSL324 systemic exposure and dose. Repeat CSL324 doses produced a sustained increase in serum G-CSF, with peak concentrations occurring 2 days after each dose. G-CSF was not detectable in cerebrospinal fluid after a single 0.8 mg/kg CSL324 dose.

Serum concentrations of cytokines and chemokines showed no clear patterns over time after CSL324 dosing in comparison with placebo. Serum interleukin (IL)-8 concentrations showed small increases with CSL324 and placebo, suggesting an effect of the IV infusion. Serum IL-1 receptor antagonist (IL-1RA) levels increased after G-CSF challenge and then decreased after administration of the higher CSL324 doses (0.3 to 1.0 mg/kg).

### Safety

Overall, the frequency of TEAEs was similar with CSL324 (82.1%) and placebo (94.7%). Treatment-related TEAEs occurred for 64.1% of subjects in the overall CSL324 group and 57.9% in the placebo group. TEAEs that occurred more frequently with CSL324 than with placebo were Neutropenia (19.2% versus no subjects), Infusion site pain (7.7% versus no subjects), and Nasal congestion (7.7% versus no subjects). No TEAEs were serious or fatal. Two subjects did not receive Dose 3 at the recommendation of the SRC.

There was no CSL324 dose-dependent trend in overall TEAE frequency across dose cohorts. All subjects (100%) experienced TEAEs after repeat dosing with CSL324 or placebo.

The majority of TEAEs were Grade 1 or 2. All treatment-related TEAEs after CSL324 treatment had resolved by the Safety Follow-up Visit, except for one TEAE of Grade 2 Erythema which was ongoing.

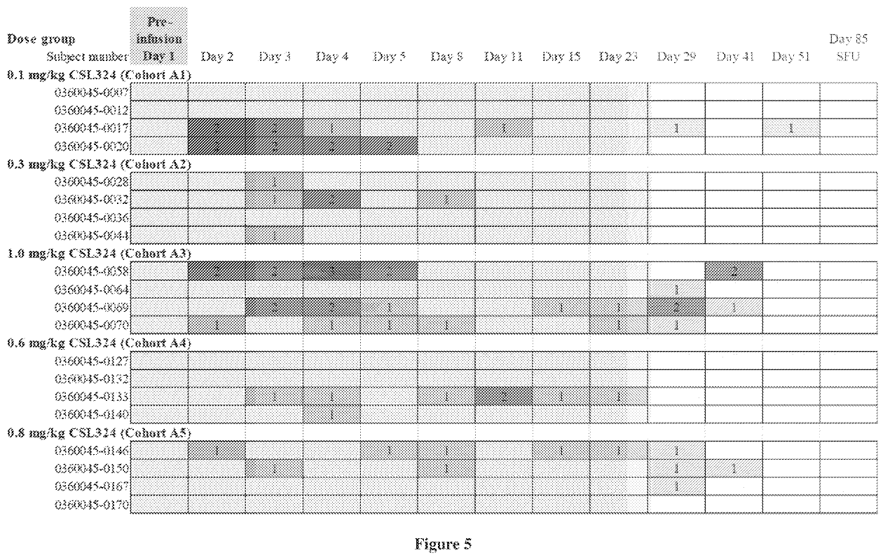

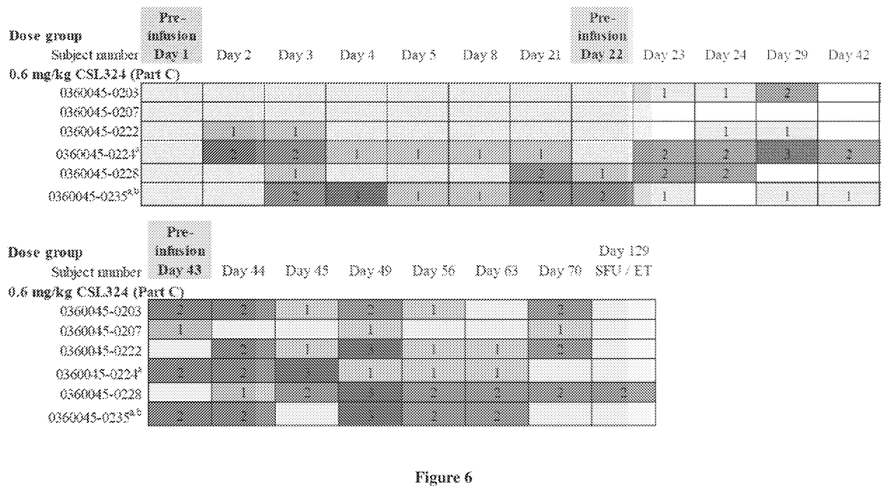

CSL324 reduced ANC in a dose-dependent manner, characterized by neutropenia up to Grade 3 severity, which resolved spontaneously the following day (FIG. 5 and FIG. 6).

One subject had a TEAE of Grade 3 Neutropenia on Day 4 after a single dose of 1.0 mg/kg CSL324 (FIG. 5) and 4 subjects had 7 TEAEs of Grade 3 Neutropenia with repeat 0.6 mg/kg CSL324 doses (FIG. 6). Two subjects who experienced more than 1 event of Grade 3 Neutropenia did not receive CSL324 Dose 3 in Part C at the recommendation of the SRC after review of available safety, tolerability, PK, and selected PD data. All TEAEs of Grade 3 Neutropenia resolved spontaneously without treatment by the next day.

ANCs meeting the criteria for neutropenia Grade 2 or 3 were experienced by 6 of 20 subjects treated with a single CSL324 dose, and tended to occur within 1 to 4 days after CSL324 dosing. Five of 6 subjects who received repeat CSL324 doses had ANCs meeting the criteria for neutropenia Grade 2 or 3, which tended to occur after Dose 3.

No infusion reactions or local tolerability reactions were observed. TEAEs of infusion site pain, puncture site erythema, and puncture site pain were experienced by <5% of CSL324-treated subjects and no placebo-treated subjects.

No safety signals were identified from laboratory parameters, vital signs including orthostatic challenge, ECG, physical findings, or fatigue scores.

No subjects developed anti-CSL324 antibodies after single and repeat IV dosing.

### CONCLUSIONS

CSL324 was safe and well tolerated when administered as a single dose up to 0.8 mg/kg or as repeat doses of 0.6 mg/kg at 21-day intervals. CSL324 reduced ANC levels in a dose-dependent manner, characterized by neutropenia up to Grade 3 severity which resolved spontaneously without treatment by the next day. Systemic CSL324 exposure increased with increasing dose, with Cₘₐₓ showing linear proportionality to CSL324 dose. Higher CSL324 doses had a longer t₁₁₂ and slower CLₜₒₜ. CSL324 showed rapid G-CSF receptor saturation and inhibited the G-CSF-mediated stimulation of ANC at higher doses, with minimal effects on inflammatory mediators.

### Example 2—Treatment of Neutrophilic Dermatosis with CSL324, an Antibody that Binds to G-CSFR

### Study Design

A multicenter, open-label two regimen repeat-dose study is used to investigate the safety and PK of repeat doses of CSL324 administered intravenously in subjects with HS and PPP. The study also investigates the preliminary efficacy of CSL324 in subjects with HS and PPP.

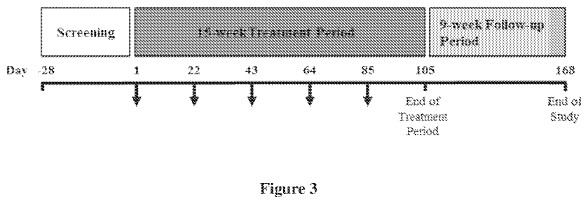

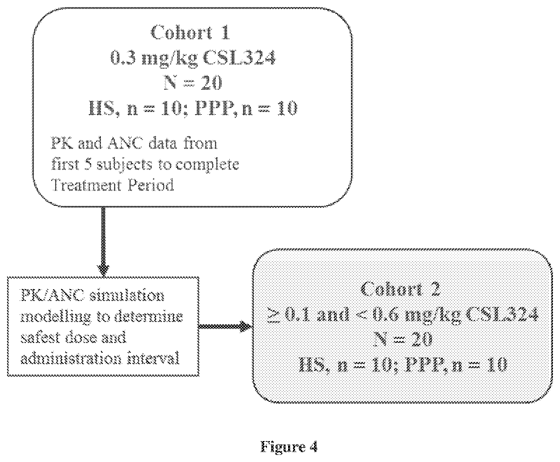

The study consists of a 28-day Screening Period, a 15-week Treatment Period, and a 9-week Follow-up Period. The study has 2 cohorts. Each cohort consists of 20 subjects with HS (n=10) or PPP (n=10) (FIG. 3). CSL324 is administered initially to subjects enrolled in Cohort #1 as a 60-minute IV infusion of 0.3 mg/kg CSL324 at 21-day intervals on Days 1, 22, 43, 64, and 85. CSL324 is administered to subjects in Cohort #2 when the first 5 subjects administered CSL324 in Cohort #1 complete the 15-week Treatment Period. The safest CSL324 dose (>0.1 and <0.6 mg/kg) for Cohort #2 is determined by PK/ANC simulation modelling, which is updated with the PK and ANC data from the first 5 Cohort #1 subjects to complete the 15-week Treatment Period (FIG. 4).

On the day of dosing, subjects remain at the study centre for at least 3 hours after the end of the infusion for safety observations and blood sampling for PK, hematology, biochemistry and cytokine/chemokine concentration and other selected biomarkers in serum. Clinical efficacy is assessed throughout the Treatment Period and Follow-up Period and tissue biopsies are collected at the start (Day 1 before CSL324 administered) and the end of the treatment period (Day 105 if all 5 doses of CSL324 are administered or 3 weeks post final dose for premature treatment cessation).

### CSL324 Antibody

TABLE 1
Antibody dose, dosing regimen, and administration

| Substance name | CSL324 |
|---|---|
| Active substance | Recombinant Anti G-CSF Receptor |
| | Monoclonal Antibody |
| Dosage form | Sterile solution for injection in 10 mL vials. |
| Dose | Cohort #1 - 0.3 mg/kg |
| | Cohort #2 - ≥0.1-≤0.6 mg/kg |
| Dosing regimen | Five doses of CSL324 will be administered |
| | every 21 days on Days 1, 22, 43, 64, and 85 |
| Mode of administration | Intravenous infusion |
| Anatomic location of | Arm |
| administration | |

### Dosing Regimen Justification

Two doses are administered to patients with HS or PPP to explore the potential PK/PD (ANC) relationship. The starting dose regimen to be tested in this study in Cohort #1 is 0.3 mg/kg every 21 days for a total of 5 doses. This starting dose regimen is selected based on safety, PK and PD data obtained in the Phase 1 study described in Example 1. Pre-clinical results in cynomolgus monkeys were also considered to support a total of 5 doses administered every 21 days.

The results from Example 1 showed that CSL324 was safe and well tolerated when administered as a single dose up to 0.8 mg/kg or as repeat doses of 0.6 mg/kg at 21-day intervals. Minimal accumulation of CSL324 was observed after 3 multiple doses of 0.6 mg/kg, with a mean (SD) terminal half-life of 251 (55.2) hrs.

Single CSL324 doses of 0.3 and 0.8 mg/kg inhibited the stimulation of ANC levels by G-CSF, confirming the mechanism of action; minimal effect was seen with the 0.1 mg/kg CSL324 dose. Following single or multiple doses of CSL324, G-CSF receptor occupancy (RO) occurred rapidly and reached ˜100% occupancy even at the lowest dose tested (0.1 mg/kg), and was sustained at this level for longer periods of time as the dose increased. Furthermore, receptor occupancy was ˜100%, for up to 27 days after administration of the third dose of 0.6 mg/kg, indicating full receptor occupancy for the dosing interval of 21 days after three doses.

Although CSL324 was safe and well tolerated, transient Grade 3 neutropenia was observed in 1 subject (1 event) following a single dose of 1 mg/kg, and 4 subjects (7 events) following three repeated doses of 0.6 mg/kg. The safest CSL324 dose (>0.1 and <0.6 mg/kg) for Cohort #2 is determined by PK/ANC simulation modelling and is supported by exposure safety margins compared to the GLP toxicology study in cynomolgus monkeys. In the GLP toxicology study (APQ0045), CSL324 was administered once weekly by slow bolus infusion for 12 weeks. CSL324 was well-tolerated in the cynomolgus monkey (both male and female), and no tested article-related toxicological findings were observed, resulting in a NOAEL of the highest dose used, 100 mg/kg. The safety margin for the proposed dose regimen in the current study was approximately 122 and 231 for AUC and Cₘₐₓ, respectively.

### Study Inclusion and Exclusion Criteria

TABLE 2
Study inclusion criteria

| Inclusion Criteria | | | Rationale | |
|---|---|---|---|---|
| 1. | For hidradenitis suppurativa: | | | |
| | a. | A confirmed clinical diagnosis of | a. | To ensure patients with confirmed |
| | | HS | | HS enroll in the study |
| | b. | Diagnosed with moderate to | b. | Disease severity enrichment of |
| | | severe HS based on IHS4 | | moderate to severe HS to optimize |
| | | guidelines (IHS4 ≥ 4) | | for assessment of treatment effect |
| | c. | The subject agrees to use | | |
| | | antiseptic wash | c. | To control inflammation associated |
| | | (Chlorhexidine 4%) daily at least | | with possible secondary infections |
| | | 2 weeks preceding Day 1 until | | |
| | | End of Study visit | | |
| 2. | For palmoplantar pustular psoriasis: | | | |
| | a. | Confirmed diagnosis of PPP, | a. | To ensure patients with confirmed |
| | | differentiated from other forms of | | PPP enroll in the study |
| | | pustulosis or psoriasis | b. | Disease severity enrichment of |
| | b. | ppPASI score of ≥12 | | ppPASI >12 to optimize for |
| | | | | assessment of treatment effect |

| | | |
|---|---|---|
| 3. | Male or female between 18 and 75 | First clinical study to assess safety in |
| | years of age, inclusive. | ND population; all diseases affect both |

males and females; it can occur at any
stage of life

TABLE 3
Study exclusion criteria

| Exclusion Criteria | | Rationale |
|---|---|---|
| 1. | Treatment with any medications and | To limit interference with evaluation of |
| | treatments listed as ‘Not Permitted’ | the study medication |
| | under Concomitant Medications and | |
| | Treatments. | |
| 2. | History of myeloproliferative disease. | To consider subjects' safety as well as |
| | | limit interference with evaluation of the |
| | | study medication and satisfactory |
| | | conduct of the study |
| 3. | Concurrent diagnosis of malignancy | To consider subjects' safety as well as |
| | (other than basal cell or squamous cell | limit interference with evaluation of the |
| | carcinoma of the skin no recurrence or | study medication and satisfactory |
| | metastases for more than 2 years prior). | conduct of the study |
| 4. | Subjects with a current or recent | To consider subjects' safety as well as |
| | clinically significant history of severe, | limit interference with evaluation of the |
| | progressive and/or uncontrolled renal, | study medication and satisfactory |
| | hepatic, hematologic, endocrine, | conduct of the study |
| | pulmonary, or cardiac disease, as | |
| | determined by the Investigator and/or | |
| | Sponsor. | |
| 5. | Subjects with immunosuppressive | To consider subjects' safety as well as |
| | conditions. | limit interference with evaluation of the |
| | | study medication and satisfactory |
| | | conduct of the study |
| 6. | Subjects who are taking | To consider subjects' safety as well as |
| | immunosuppressive or | limit interference with evaluation of the |
| | immunomodulative therapy as | study medication and satisfactory |
| | described in prohibited medication | conduct of the study |
| | section | |
| 7. | Clinical signs of active infection and/or | To consider subjects' safety as well as |
| | fever >38° C. within 7 days preceding | limit interference with evaluation of the |
| | Day 1. Study entry may be deferred for | study medication and satisfactory |
| | such individuals at Investigator and/or | conduct of the study |
| | Sponsor discretion. | |
| 8. | Subjects with a confirmed HIV, | To consider subjects' safety as well as |
| | Hepatitis B or C infection | limit interference with evaluation of the |
| | | study medication and satisfactory |
| | | conduct of the study |
| 9. | Subjects with clinically | To consider subjects' safety as well as |
| | significant laboratory | limit interference with evaluation of the |
| | abnormalities including aspartate | study medication and satisfactory |
| | aminotransferase or alanine | conduct of the study |
| | aminotransferase > 2 × upper limit | |
| | of normal or neutropenia (defined | |
| | as ANC < 2 × 10⁹/L) | |
| 10. | History of chronic alcohol or drug | To consider subjects' safety as well as |
| | abuse within previous 1 year | limit interference with evaluation of the |
| | | study medication and satisfactory |
| | | conduct of the study |
| 11. | Female subjects who are | Non-clinical reproductive studies have |
| | pregnant, breastfeeding, plan to | not been conducted |
| | become pregnant during the study | |
| | or within 30 days following | |
| | EOSV, or those of child bearing | |
| | potential not willing to use an | |
| | acceptable form of contraception | |
| | for the duration of the study. | |
| 12. | Male subjects with partners who | Non-clinical reproductive studies have |
| | are planning pregnancy during the | not been conducted |
| | study or within 30 days following | |
| | EOSV, or those whose partners of | |
| | child bearing potential not willing | |
| | to use an acceptable form of | |
| | contraception for the duration of | |
| | the study. | |
| 13. | For palmoplantar pustulosis: | Due to the many varieties of psoriasis, |
| | Concurrent background of | the exclusion of concurrent psoriasis |
| | psoriasis vulgaris. | vulgaris allows for a clear PPP |
| | | population to be enrolled in the study |
| 14. | For hidradenitis suppurativa: | Avoid patients with serious, possibly |
| | Subjects with >20 draining | untreatable disease |
| | fistulas | |

### Safety Assessments

The clinical procedures conducted during this study related to the evaluation of safety are provided in Table 4 below.

TABLE 4
Safety assessments

| Assessment | Description | | |
|---|---|---|---|
| Pregnancy/FS H | Serum test for Choriogonadotropin Beta (beta-human chorionic | | |
| test | gonadotropin [β-hCG]) to test for pregnancy | | |
| | Follicle stimulating hormone (FSH) to confirm menopause | | |
| Physical | As per the site's standard procedure | | |
| examination | | | |
| 12-lead ECG | Heart Rate | PR Interval | QRS Duration |
| | QT Interval | QTcB Interval | QTcF Interval |

| | Interpretation (investigator's overall interpretation) | |
|---|---|---|
| Adverse events | Evaluation of all adverse events (eg, causality/relatedness, | |
| | severity, seriousness) | |
| | Adverse events of special interest: | |
| | Gr3 (Severe)/Gr4 (life threatening) neutropenia | |
| | Infections | |
| Vital signs | Blood Pressure (Systolic and | Temperature |
| | Diastolic) | Height |
| | Respiratory Rate | Weight |
| | Pulse Rate | |

| | | | |
|---|---|---|---|
| Urinalysis | Specific Gravity | Nitrite | Protein |
| (dipstick) | pH | Ketones | Glucose |
| | Eeukocyte Esterase | Bilirubin | |
| | Occult Blood | Urobilinogen | |

| Haematology | Leukocytes (White blood cell [WBC] Count) | |
|---|---|---|
| | Hemoglobin (HGB) Hematocrit (HCT) | |
| | Erythrocytes (Red Blood Cell [RBC] Count) | |
| | Red blood cell indices: Mean Corpuscular Volume (MCV); Mean | |
| | Corpuscular Hemoglobin (MCH); Mean Corpuscular | |
| | Hemoglobin Concentration (MCHC); Erythrocyte Distribution | |
| | Width (RDW) | |
| | Platelets | |
| | Differential - percentage and absolute: Neutrophils; Neutrophil | |
| | Band Forms; Lymphocytes; Monocytes; Eosinophils; Basophils | |
| | Reticulocytes | |
| Biochemistry | Sodium (Na) | Aspartate Aminotransferase |
| | | (AST) |
| | Potassium (K) | Lactate Dehydrogenase |
| | | (LDH) |
| | Chloride (Cl) | Gamma-Glutamyl |
| | | Transferase (GGT) |
| | Bicarbonate (HCO₃) | Bilirubin - total |
| | Carbon Dioxide - total (CO₂) | Direct Bilirubin |
| | Calcium (Ca) | Magnesium (Mg) |
| | Urea Nitrogen (BUN) | Phosphate (PO₄) |
| | Urea | C Reactive Protein (CRP) |
| | Creatinine | Cholesterol - total |
| | Glucose | Triglycerides |
| | Protein - total | HDL Cholesterol |
| | Albumin | LDL Cholesterol |
| | Alkaline Phosphatase | Urate (Uric Acid) |
| | Alanine Aminotransferase (ALT) | Creatinine Kinase |
| | | (CK; CPK) |
| Immunogenicity | Serum analysed for the presence of binding antibodies to CSL324 | |

### Absolute Neutrophil Count (ANC) and Body Temperature Monitoring

Each subject's ANC is monitored throughout the study at scheduled time points.

If a subject records a Grade 3 or Grade 4 neutropenia on the day before a dose administration, a repeat ANC assessment is conducted within 24 hours and the average of the 2 ANC values must be >800/mm³ to allow the dose of CSL324 to be administered. If the average of the 2 ANC values is <800/mm³, the subject does not receive any further dosing. In order to allow for repeat ANC measure to be performed in response to either Grade 3 or Grade 4 neutropenia the day prior to dosing, a dose may be delayed within the permissible dosing window (+3 days).

If a subject records a Grade 3 (Gr3) neutropenia on any other day other than the day before dosing, the subject may continue in the study unless this single Gr3 ANC value is coupled with a single tympanic temperature of >38.3° C. or >38.0° C. sustained for >1 hour and/or clinically significant signs or symptoms of infection. If a subject has a Grade 3 neutropenia at any other time during the Treatment or Follow-up periods, unscheduled ANC measures may be performed to monitor, as closely as feasible, the subject's ANC levels.

If a subject records a Grade 4 neutropenia on any other day other than the day before dosing, a repeat ANC assessment must be conducted within 24 hours and the subject can continue in the study if their repeat ANC value is >500/mm³. If the repeat ANC value is <500/mm³, the subject does not receive any further dosing.

Subjects with Grade 3 or 4 neutropenia are requested to be vigilant of and immediately report any signs and/or symptoms of infection including elevated body temperature. All study subjects are provided with a thermometer and are asked to monitor oral temperature at a consistent time daily. Subjects re asked to contact the site immediately if their oral temperature measure exceeds 37.2° C. and are asked to attend an unscheduled visit for clinical evaluation.

### Efficacy Assessments

### Hidradenitis Suppurativa

Total abscess and inflammatory nodule count (AN count): A nodule (inflammatory nodule) is a raised, three-dimensional, round, infiltrated lesion with a diameter of >10 mm. An abscess is a tender but fluctuating mass with a diameter of >10 mm, surrounded by an erythematous area; the middle of an abscess contains pus. A draining tunnel/fistula is a raised, tender but fluctuating longitudinal mass of variable length and depth, ending at the skin surface, and sometimes oozing fluid (Lipsker et al. 2016, Dermatology 232: 137-42). The AN count, coupled with an assessment of draining tunnels/fistulas, is assessed at screening, prior to dosing on days 1, 22, 43, 64 and 85, as well as day 105, 126, 147 and 168 (EOSV), for different scores to assess dynamic changes in HS including:

Hidradenitis Suppurativa Clinical Response (HiSCR): The HiSCR was developed and validated in 2014 to improve sensitivity, measurement consistency and ease of use (Kimball et al. 2014, Br J Dermatol 171: 1434-42). The HiSCR is a valid, responsive and meaningful clinical endpoint of inflammatory manifestations of HS that can be adapted to clinical research and daily practice. It is defined as a 50% reduction from baseline in the total AN count, with no increase in abscesses or draining fistula count. This measure has been used in several Phase 2 HS studies (Kimball, Sobell, et al. 2016, J Eur Acad Dermatol Venereol 30: 989-94; Kanni et al. 2018, J Invest Dermatol 138: 795-801; Tzanetakou et al. 2016, N Engl J Med 320: 365-76) and the PIONEER HS Phase 3 clinical studies (Kimball, Okun, et al. 2016, N Engl J Med 375: 422-34).

International Hidradenitis Suppurativa Severity Score System (IHS4): The IHS4 is a validated tool for the dynamic severity assessment of HS (Zouboulis et al. 2017, Br J Dermatol 177: 1401-09) and improves upon the HiSCR assessment as it is designed to assess treatment response rather than disease severity cross-sectionality (Kimball et al. 2014, Br J Dermatol 171: 1434-42). The IHS4 score (points)=(number of nodules multiplied by 1)+(number of abscesses multiplied by 2)+[number of draining tunnels (fistulae/sinuses) multiplied by 4]. A score of 3 or less signifies mild HS, a score of 4-10 signifies moderate HS and a score of 11 or higher signifies severe HS (Zouboulis et al. 2017, Br J Dermatol 177: 1401-09).

Hidradenitis Suppurativa Physician Global Assessment (HS-PGA): The six-point HS-PGA is used in clinical trials to measure clinical improvement in inflammatory nodules, abscesses and draining fistulae. It ranges from clear (Score 0) to very severe (Score 5). It has clear guidance for disease severity scoring and is relatively easy to use (Kimball et al. 2014, Br J Dermatol 171: 1434-42). HS-PGA will be assessed pre-dose on day of dosing (Days 1, 22, 43, 64 and 85), Day 105, 126, 147 and 168 (EOSV).

### Palmoplantar Pustulosis

Palmoplantar Pustulosis Psoriasis Area Severity Index (ppPASI): ppPASI is an assessment tool based on the Psoriasis Area and Severity Index that is widely used for assessing severity of chronic plaque psoriasis. Parameters including severity, erythema, total number of pustules and desquamation are scored on a scale of 1-4, then corrected for area and site involved (palm or sole). The sum of the four values produces the final ppPASI which ranges between 0 (no PPP) and 72 (the most severe PPP) (Bhushan et al. 2001, Br J Dermatol 145: 546-53). ppPASI is assessed at screening, prior to dosing on days 1, 22, 43, 64 and 85, as well as day 105, 126, 147 and 168 (EOSV).

Palm-Sole Physician Global Assessment (PGA) score: PGA is an average assessment of all psoriatic lesions based on erythema, scale, and induration (Robinson, 2011). PGA is assessed pre-dose on day of dosing (Days 1, 22, 43, 64 and 85), Day 105, 126, 147 and 168 (EOSV).

### Pharmacokinetic (PK) Assessments

Following the first and last infusions, PK samples for determination of serum concentrations of CSL324 are collected from a contralateral arm (in respect to i.v. line for infusion) by vein puncture prior to each infusion and at end of infusion, and at 3 hrs, 4 days, 1 week, 2 weeks and 3 weeks after the end of infusion. Additional PK samples at 6 weeks, 9 weeks and 12 weeks after the last dose are collected.

The serum concentrations are listed by time point and summarized descriptively. Graphical displays of CSL324 PK parameters after repeated dose administration, derived by non-compartmental method, are summarised descriptively by dose cohort and indication group.

### Pharmacodynamic (PD) Assessments

Blood and tissue samples are collected for various assessments. The blood assessments include, but are not limited to the following: Serial ANC measurements, serum cytokines and chemokines (eg. G-CSF, GM-CSF), disease associated pro-inflammatory markers (eg. CRP, ESR, C3a, C5a), inflammatory gene signature(s) (eg. Neutrophil/G-CSF signature) and neutrophil profile shift based on peripheral blood smears. Extra blood draws for research analysis are clearly mentioned in the informed consent form (ICF) with specific approval required for their collection.

### Skin Biopsy Collection

Tissue samples, via punch biopsies, are collected at Baseline and 3 weeks after the final dose to assess cellular infiltration, including but not limited to neutrophil infiltration. This analysis is done by histology (Immunohistochemisty, H&E) and RNA assessment.

Two×3 mm biopsies are collected each on Day 1 (Baseline) and at the end of the treatment period (Day 105 if all 5 doses of CSL324 are administered or 3 weeks post final dose for premature treatment cessation). Biopsies are collected prior to dosing and blood collection, and after vital signs, temperature and clinical endpoint assessments. The end of study treatment biopsies are taken as close as possible to the Day 1 biopsy site even if the lesion has partially or completely cleared. For HS, baseline biopsies are collected directly from a nodule >1 cm (largest nodule possible) avoiding the center of the nodule if possible. For PPP, baseline biopsies are collected from an area of inflamed skin on the palm of the hand or the sole of the feet near, but not including, a pustule.

### Patient Reported Outcome Assessments

### All Diseases:

Dermatology Quality of Life Index questionnaire (DLQI) score: The DLQI is a simple 10-question validated questionnaire that has been used in over 40 different skin conditions in over 80 countries and is available in over 90 languages. Its use has been described in over 1000 publications including many multinational studies. Each question is scored from 0 (not at all) to 3 (very much) with the recall period being 1 week. A total of 30 points is the maximum score, where 0-1 is regarded as no effect, 2-5 small, 6-10 moderate, 11-20 very large and 21-30 as extremely large effect on the patient's life (Hongbo et al. 2005, J Invest Dermatol 125: 659-64). DLQI is assessed prior to dosing on days 1, 22, 43, 64 and 85, as well as day 105, 126, 147 and 168 (EOSV).

### Hidradenitis Suppurativa:

Numerical Rating Scale (NRS) pain score: The NRS for pain is a unidimensional measure of pain intensity in adults, including those with chronic pain associated with dermatological conditions (Kimball, Okun, et al. 2016, N Engl J Med, 375: 422-34). The NRS is a segmented numeric version of the visual analog scale (VAS) in which a respondent selects a whole number (0-10 integers) that best reflects the intensity of their pain over the last 24 hours (Rodriguez 2001, Pain Manag Nurs, 2: 38-46). The common format is a horizontal bar or line and is anchored by terms describing pain severity extremes, (Hawker et al. 2011, Arthritis Care Res (Hoboken), 63 Suppl 11: S240-52). The NRS pain score is collected daily using an electronic diary, with weekly averages derived.

### Other Assessments

Photographs capturing lesion changes over time is an optional assessment for subjects to participate in. These photographs are taken at Baseline as well as various times over the course of the study to capture lesion with CSL324 treatment (Week 3, 6, 9, 12, 15 and follow-up). These photographs may be used in various settings and documents including internal and external presentations, reports and publications.

### Stopping Rules

### Study Stopping Criteria

The study is stopped immediately if:
One subject develops a serious AE (SAE) that results in death and considered by the Investigator and/or Sponsor to be related to the administration of CSL324. If any of the following events occur during the study and these events are considered to be related to the administration of CSL324, recruitment is stopped and the event is investigated to determine recommending stopping the study, modifying the protocol before restarting the study, or restarting the study:
If any of Cohort stopping criteria are met in the lower dose cohort
One or more subjects developing any other event that is deemed to pose an unacceptable risk to other subjects in the study.

### Cohort Stopping Criteria

During this study, safety-related cohort stopping criteria is in place for all indications. If Cohort stopping criteria are met in the higher dosing cohort, the lower dose cohort can be continued unless the SRC recommends a study stop.

Dosing of all subjects in an individual Cohort is stopped immediately if:
Three (3) subjects within one Cohort have a single event of Grade 4 neutropenia related to CSL324 administration (defined as a single measurement confirmed on repeat measurement approximately 24 hours later).
If any of the following events occur during the study in subjects within one cohort and these events are considered to be related to the administration of CSL324, the event is investigated to provide a recommendation for allowing further dosing and recruitment for the affected cohort. It is also decided whether to continue the dosing of study subjects within the same cohort who have not met the below criteria and who are part-way through their treatment period, or to stop all dosing of CSL324 for the entire cohort and have all cohort subjects undergo end of treatment and follow up assessments and return to clinical care/SoC.
   One subject has a single event of Grade 4 neutropenia (confirmed on repeat measurement approximately 24 hours later) in the presence of any clinical signs or symptoms of infection.
   One subject has confirmed neutropenic sepsis, requiring IV antibiotics (Criteria to define sepsis TBD).
   Any other event that is deemed to pose an unacceptable risk to other subjects in the cohort.

### Subject Stopping Criteria

During the study, safety-related subject stopping criteria are in place for all indications. If a subject develops any of the following events during the study and the event(s) is considered to be related to CSL324, the subject is not administered any remaining doses of CSL324.
A serious adverse event (SAE).
If the subject experiences prolonged symptoms of a Grade 3 or 4 infusion reaction (per CTCAE grading) despite management including slowing of infusion rate and/or administration of oral anti-histamines
A severe non-serious AE (including infections) that is considered clinically significant.
The subject experiences a single event of Grade 4 neutropenia (confirmed on repeat measurement approximately 24 hours later) at any stage during the treatment period of the study.
If the subject records a Grade 3 or Grade 4 neutropenia on the day before a dose administration and with a repeat ANC assessment (confirmed on repeat measurement approximately 24 hours later) averages <800/mm³.
Grade 3 neutropenia according to CTCAE coupled with a single tympanic temperature of >38.3° C. or >38.0° C. sustained for >1 hour and/or clinically significant signs or symptoms of infection.

In the event that a subject does not receive their complete full dosing regimen of CSL324, they return to clinical care and undergo end of treatment assessment (3 weeks post last dose) and the follow up assessment.

### Example 3—CSL324 Reduces Neutrophil Migration Associated with CXCR1 Expression, which is a Marker that is Upregulated in HS Patients

### CXCR1 Expression in HS Patients

Whole blood samples from patients with Hidradenitis suppurativa (HS; n=15) were used to assess the levels of CXCR1, a cell migration marker, on the surface of neutrophils (defined by high side scatter (SSC), CD11b+CD49−) by flow cytometry compared to neutrophils from age and sex-matched healthy control whole blood samples.

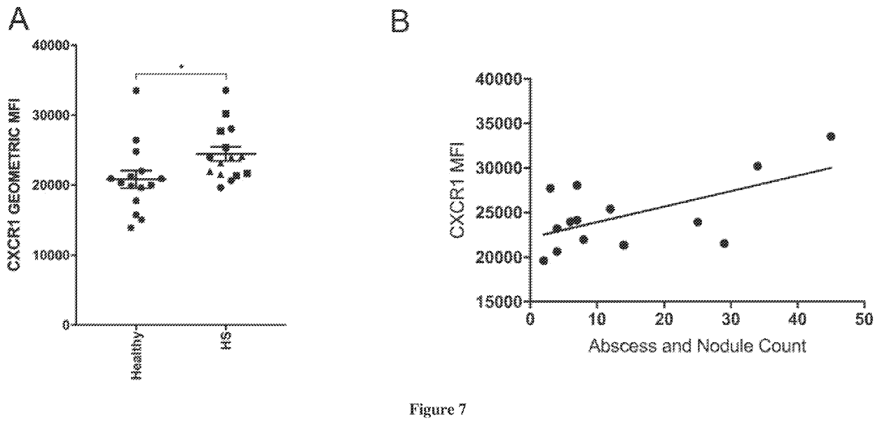

As shown in FIG. 7A, CXCR1 expression was significantly higher in HS patient sample neutrophils compared to healthy controls (n=15). Further analysis demonstrated a correlation between HS patient abscess and nodule count, a form of disease activity assessment, and CXCR1 expression on neutrophils in HS patients (n=14) was observed (FIG. 7B; r²=0.3532, p=0.0250).

### CSL324 Reduces CXCR1 and CXCR2 Expression Induced by G-CSF

Whole blood samples obtained from healthy human donors were used to assess the expression of chemokine receptors CXCR1 and CXCR2 on neutrophils and to assess the effects of CSL324 in the presence or absence of G-CSF on the levels of these migratory receptors. Samples were pre-incubated with 1 mg/mL of CSL324 for 30 minutes prior to the stimulation of the cells with recombinant human G-CSF (30 ng/mL; n=11) or recombinant human GM-CSF (30 ng/mL; n=4) and cultured for 20 hours at 37° C., 5% CO₂. Neutrophils were identified by high side scatter (SSC) and the CD11b+CD49d− phenotype. The mean fluorescence intensity of conjugated antibodies to CXCR1 or CXCR2 was normalized relative to cells cultured in media alone.

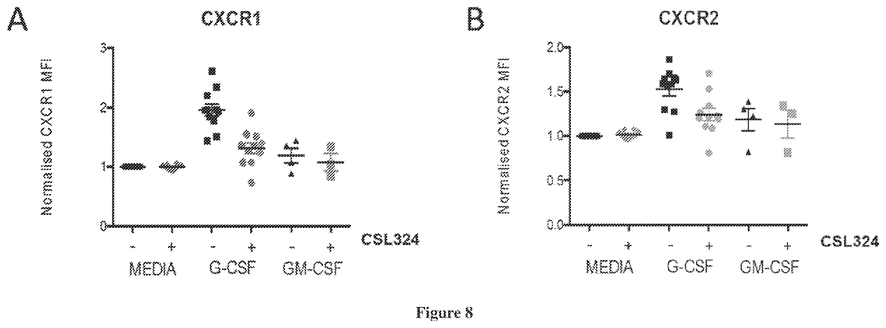

As shown in FIG. 8, culture of neutrophils with G-CSF alone (black) increased the cell surface expression of CXCR1 and CXCR2 compared to media alone. Pre-incubation with CSL324 (grey) caused a reduction in the G-CSF induced up-regulation of CXCR1 and CXCR2, with the mean fluorescence intensity (MFI) of CXCR1 or CXCR2 staining comparable to that seen when neutrophils were incubated in cell culture media alone. Culture of cells in the presence of GM-CSF did not significantly alter the levels of surface markers, and further, was not altered by the pre-incubation of samples with CSL324.

### CSL324 Reduces Cell Migration Induced by G-CSF

A cell migration assay was used to assess the ability of CSL324 to inhibit G-CSF mediated neutrophil migration towards MIP-2. Specifically, purified neutrophils were isolated (>95% purity) and pre-cultured with or without 1 μg/mL CSL324 for 30 minutes before being stimulated with 30 ng/mL human G-CSF or 30 ng/mL human GM-CSF overnight. Chemotaxis to MIP-2 (500 ng/mL) was measured using transwell inserts (5 μm pores).

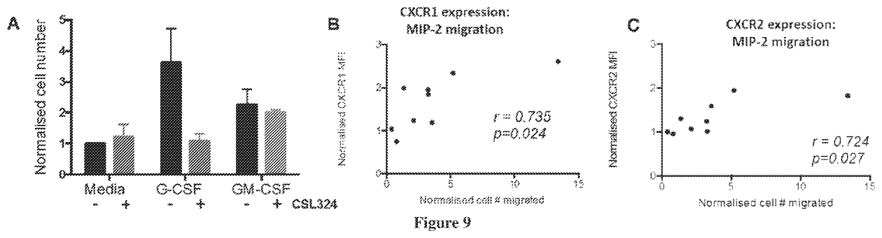

As shown in FIG. 9, pre-incubation with G-CSF resulted in increased migration of neutrophils to MIP-2, which was reduced to the same levels as the media alone condition by pre-incubation with CSL324 (FIG. 9A; grey bars). The pro-migratory effects of GM-CSF were not inhibited by pre-incubation with CSL324, indicating specificity to the effects of engaging the G-CSF receptor. Pre-incubation with G-CSF resulted in up-regulation of CXCR1 and CXCR2 that correlated with increased migration of neutrophils to MIP-2 (FIGS. 9B and 9C).

Together, these data demonstrate that:
CXCR1 is expressed at significantly higher levels on neutrophils in HS patients relative to healthy individuals (FIG. 7A);
CXCR1 expression is positively correlated with severity of HS disease (as measured by abscess and nodule count; FIG. 7B);
CXCR1 (and CXCR2) expression is positively correlated with neutrophil migration (FIGS. 9B and 9C);
CSL324 inhibits G-CSF-induced CXCR1 (and CXCR2) expression on neutrophils (FIGS. 8A and 8B) as well as G-CSF-induced neutrophil migration (FIG. 9A).

### Example 4—Neutrophil Numbers and Migration Marker Expression are Upregulated in Psoriasis Patients

To assess the potential for treatment with an antibody that binds to G-CSFR, psoriasis patients were assessed for their neutrophil numbers in whole blood and expression cell migration markers CXCR1 and CXCR2.

A total of 21 individuals with plaque psoriasis (also known as “psoriasis vulgaris” or “common psoriasis”) and 21 age and sex-matched unaffected individuals were recruited for collection of blood and skin tissue samples. Neutrophils in fresh whole blood samples were phenotyped using flow cytometry.

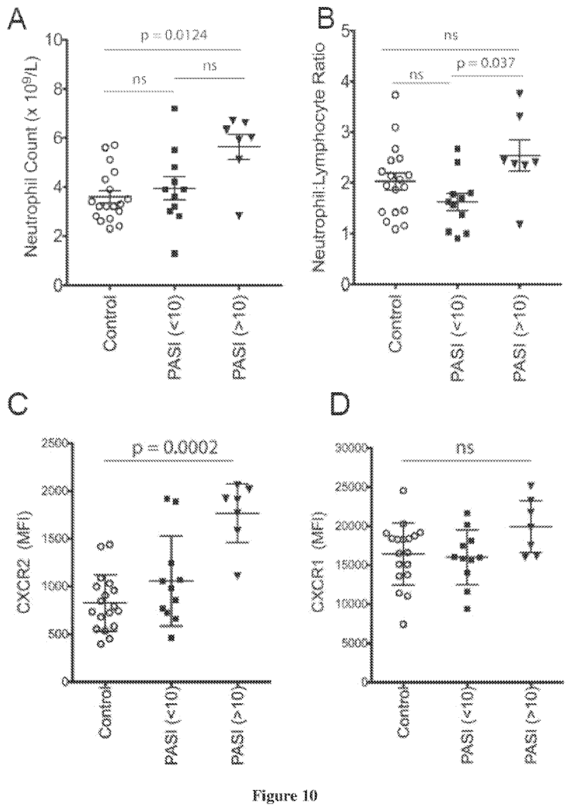

As shown in FIG. 10, neutrophil counts (FIG. 10A) were significantly increased in the peripheral blood of people with psoriasis compared to unaffected controls. Stratification based on the severity of psoriasis as assessed by PASI score showed that neutrophil counts were significantly elevated in individuals with a PASI score of 10 or greater. Furthermore, The neutrophil:lymphocyte ratio (NLR) was significantly elevated in individuals with a PASI score of 10 or greater compared to individuals with a PASI score of less than 10 (FIG. 10B).

Expression of cell surface markers of activation and migration, CXCR1 and CXCR2, were assessed on peripheral blood neutrophils in people with psoriasis compared to unaffected controls by flow cytometry. The chemokine receptor CXCR2 was significantly elevated on the surface of neutrophils in both mild (PASI <10) and severe (PASI >10) psoriasis (FIG. 10C). No significant alteration in the levels of the chemokine receptor CXCR1 was detected (FIG. 10D). Given that an antibody which binds to G-CSFR, CSL324, has been demonstrated herein to reduce neutrophil count (see Example 1) and expression of CXCR1 and CXCR2 (see Example 3), these data provide evidence that such an antibody may be a viable therapeutic option for treatment of psoriasis.

### Example 5—Treatment of Palmoplantar Pustulosis (PPP) with CSL324 is Safe and Efficacious

### Subject 00360098-0001

The subject was a 52 year old Caucasian human male with a history of palmoplantar pustulosis (PPP) for over four years, obesity (129 kg), and smoking (for over 30 years). He was treated previously for PPP with the following medications:
Methotrexate 10 mg weekly for approximately one year;
Acitretin 50 mg daily for approximately four months;
Tacrolimus (topical) for approximately three years;
Corticosteroid (topical) for approximately three years; and
Vitamin D+corticosteroids (topical) for approximately three years.

The subject was enrolled into Study CSL324_1002 described in Example 2 and received his first infusion of CSL324 at a dose of 0.3 mg/kg on day 1 and then received 4 subsequent infusions every 21 days on days 22, 43, 64, and 85.

### Efficacy Results

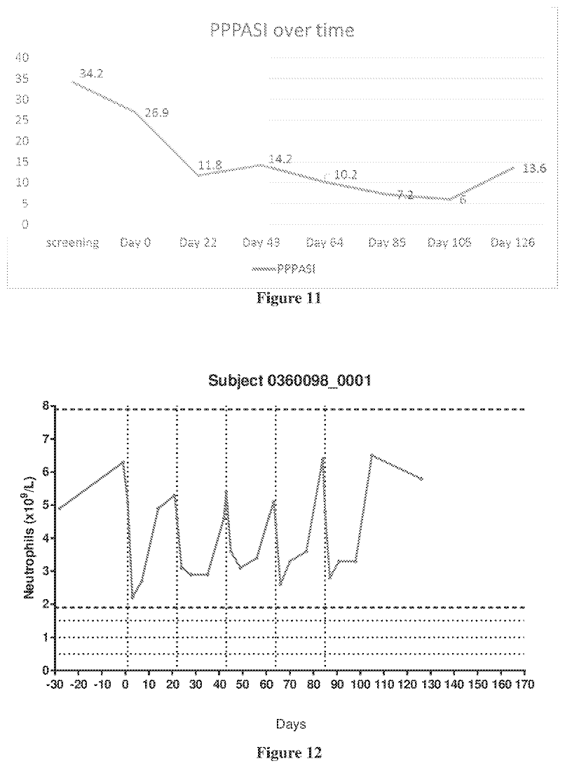

The subject's baseline PPP severity, as measured by the Palmoplantar Pustular Psoriasis Area Severity Index (ppPASI), was 34.2 (severe) at screening and 26.9 (severe) prior to first administration of CSL324. In addition, prior to first administration of CSL324, the PPP-Physician's Global Assessment (PPP-PGA) was severe. The results of efficacy measures through day 126 are presented in Table 5 and FIG. 11.

TABLE 5
ppPASI and PPP-PGA scores for Subject 00360098-0001

| | | Day | Day | Day | Day | Day | Day | Day |
|---|---|---|---|---|---|---|---|---|
| | Screening | 0 | 22 | 43 | 64 | 85 | 105 | 126 |
| ppPASI | 34.2 | 26.9 | 11.8 | 14.2 | 10.2 | 7.2 | 6 | 13.6 |
| PPP-PGA | | severe | moderate | moderate | moderate | mild | mild | moderate |

The data presented in Table 5 and FIG. 11 demonstrates that treatment with CSL324 successfully reduces the severity of PPP, as measured by ppPASI or PPP-PGA. Table 6 below provides a guideline for interpreting the results.

TABLE 6
interpretation of CSL324 efficacy assessment
ppPASI

| | |
|---|---|
| ppPASI < 7 | Mild |
| ppPASI = 7-15 | Moderate |
| ppPASI > 15 (up to 72) | Severe |

PPP-PGA

| | |
|---|---|
| 0 | Clear |
| 1 | almost clear |
| 2 | mild |
| 3 | moderate |
| 4 | severe |

### Safety Results

### Adverse Events

The subject experienced 3 adverse events, all non-serious and considered not related to CSL324. The first AE, lower back pain, occurred on Day 67, was treated with an NSAID and resolved in 1 day. The second AE, diabetes mellitus, was diagnosed on the day of the final dose of CSL324, after having elevated glucoses since study screening that did not change despite dietary modifications. Hyperglycemia did not worsen during treatment with CSL324. The final AE, lethargy, occurred the day after the final dose and resolved the same day.

### Absolute Neutrophil Count

The subject had an absolute neutrophil count (ANC) of 4.9×10⁹/L and 6.3×10⁹/L prior to first administration of CSL324. The ANC remained in the normal range throughout the study, as illustrated by FIG. 12 and Table 7 below.

1. A method for reducing circulating neutrophils in a human subject without causing sustained grade 3 or grade 4 neutropenia for greater than seven consecutive days, the method comprising administering to the subject a dose of between 0.1 mg/kg and 1.0 mg/kg of an antibody that inhibits G-CSF signaling.

2. The method of claim 1, wherein the subject suffers from a neutrophil-mediated condition.

3. A method for treating a neutrophil-mediated condition, the method comprising administering to a subject suffering from the neutrophil-mediated condition a dose of between 0.1 mg/kg and 1.0 mg/kg of an antibody that inhibits G-CSF signaling.

4. The method of claim 3, wherein administration of the antibody does not cause sustained grade 3 or grade 4 neutropenia in the subject for greater than seven consecutive days.

5. The method of claim 1, wherein
i) administration of the antibody does not induce grade 4 neutropenia;
ii) administration of the antibody does not induce neutropenia or the antibody induces grade 2 or grade 3 neutropenia for two consecutive days or less;
iii) administration of the antibody does not induce neutropenia for more than 2 consecutive days or more than 1 day; or
iv) the neutropenia is not associated with a fever.

6.-23. (canceled)

24. The method of claim 1, wherein the antibody is administered at a dose of between 0.1 mg/kg and 0.6 mg/kg.

25. The method of claim 1, wherein the antibody is administered at a dose of 0.1 mg/kg or 0.3 mg/kg or 0.6 mg/kg.

26. The method of claim 1, wherein the antibody is administered multiple times, wherein the antibody is administered once every 14 to 28 days.

27. The method of claim 1, wherein the antibody is administered multiple times, wherein the antibody is administered once every 21 days.

28. The method of claim 1, wherein the antibody binds to G-CSFR and inhibits G-CSF signalling.

29. The method of claim 1, wherein the antibody binds to or specifically binds to granulocyte-colony stimulating factor receptor (G-CSFR) and competitively inhibits the binding of antibody C1.2G comprising a heavy chain variable region (V_{H}) comprising a sequence set forth in SEQ ID NO: 4 and a light chain variable region (V_{L}) comprising a sequence set forth in SEQ ID NO: 5 to G-CSFR.

30. The method of claim 1, wherein the antibody binds to an epitope comprising residues within one or two or three or four regions selected from 111-115, 170-176, 218-234 and/or 286-300 of SEQ ID NO: 1.

31. The method of claim 1, wherein the antibody comprises:
(i) a heavy chain variable region (V_{H}) comprising an amino acid sequence set forth in SEQ ID NO: 4 and a light chain variable region (V_{L}) comprising an amino acid sequence set forth in SEQ ID NO: 5;
(ii) a V_{H} comprising an amino acid sequence set forth in SEQ ID NO: 2 and a V_{L} comprising an amino acid sequence set forth in SEQ ID NO: 3;
(iii) a V_{H} comprising three CDRs of a V_{H} comprising an amino acid sequence set forth in SEQ ID NO: 4 and a V_{L} comprising three CDRs of a V_{L} comprising an amino acid sequence set forth in SEQ ID NO: 5; or
(iv) a V_{H} comprising three CDRs of a V_{H} comprising an amino acid sequence set forth in SEQ ID NO: 2 and a V_{L} comprising three CDRs of a V_{L} comprising an amino acid sequence set forth in SEQ ID NO: 3.

31. The method of claim 1, wherein the antibody comprises:
(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.

32. The method of claim 2, wherein the neutrophil-mediated condition is an autoimmune disease, an inflammatory disease, cancer or ischemia-reperfusion injury.

33. The method of claim 32, wherein the autoimmune disease or the inflammatory disease is arthritis, uveitis, multiple sclerosis, pulmonary inflammation, chronic obstructive pulmonary disease, psoriasis, or severe asthma.

34. The method of claim 2, wherein the neutrophil-mediated condition is a neutrophilic dermatosis or a neutrophilic skin lesion.

35. The method of claim 34, wherein the neutrophilic dermatosis is selected from the group consisting of amicrobial pustulosis of the folds (APF); plaque psoriasis; CARD14-mediated pustular psoriasis (CAMPS); cryopyrin associated periodic syndromes (CAPS); deficiency of interleukin-1 receptor (DIRA); deficiency of interleukin-36 receptor antagonist(DIRTA); hidradenitis suppurativa (HS); palmoplantar pustulosis; pyogenic arthritis; pyoderma gangrenosum and acne (PAPA); pyoderma gangrenosum, acne, and hidradenitis suppurativa (PASH); pyoderma gangrenosum(PG); skin lesions of Behcet's disease; Still's disease; Sweet syndrome; subcorneal pustulosis (Sneddon-Wilkinson); pustular psoriasis; palmoplantar pustulosis; acute generalized exanthematic pustulosis; infantile acropustulosis; synovitis, acne, pustulosis; hyperostosis and osteitis (SAPHO) syndrome; bowel-associated dermatosis-arthritis syndrome (BADAS); neutrophilic dermatosis of the dorsal hands; neutrophilic eccrine hidradenitis; erythema elevatum diutinum; and Pyoderma gangrenosum.

36. The method of claim 34, wherein the neutrophilic dermatosis is hidradenitis suppurativa (HS) or palmoplantar pustulosis (PPP).

37. A method for treating a neutrophilic dermatosis, the method comprising administering to a subject suffering from a neutrophilic dermatosis a dose of between 0.1 mg/kg and 1 mg/kg of an antibody that binds to or specifically binds to granulocyte-colony stimulating factor receptor (G-CSFR), wherein the antibody is administered multiple times once every 21 days and wherein the antibody comprises:
(i) a heavy chain comprising a sequence set forth in SEQ ID NO: 14 and a light chain comprising a sequence set forth in SEQ ID NO: 15; or
(ii) a heavy chain comprising a sequence set forth in SEQ ID NO: 16 and a light chain comprising a sequence set forth in SEQ ID NO: 15.
